(19) United States
(12) Patent Application Publication
(10) Pub. No.: US 2026/0014336 A1
(43) Pub. Date: Jan. 15, 2026

(54) DIGITAL TWIN AND ARTIFICIAL INTELLIGENCE (AI) MODELS FOR PERSONALIZATION AND MANAGEMENT OF BREATHING ASSISTANCE

(71) Applicant: NOVARESP TECHNOLOGIES INC., Halifax (CA)

(72) Inventors: Hamed Hanafialamdari, Halifax (CA); Scott Corren Lowe, Sandbach (GB); Guillermo Aristi Suarez, Halifax (CA); Gregory Eric Avard Begin, Halifax (CA); Brendan Ian Liam Brady, Halifax (CA); Sarah Elizabeth Reeve, Halifax (CA); Meagan Mackenzie Sinclair, Halifax (CA); Joel Liederman, Thornhill (CA); Seyedehnajmeh Sadatnejad, Beechville (CA); Kazi Antor Hasan, Halifax (CA); Rajwant Singh Sodhi, Halifax (CA)

(21) Appl. No.: 19/331,200

(22) Filed: Sep. 17, 2025

Related U.S. Application Data
(63) Continuation of application No. PCT/CA2024/050324, filed on Mar. 18, 2024.
(60) Provisional application No. 63/453,093, filed on Mar. 18, 2023.

(51) Int. Cl. A61M 16/00; A61B 5/00; A61B 5/08; A61M 16/06; G06F 18/10; G06F 30/27; G06N 3/092; G16H 50/50

(52) U.S. Cl. CPC A61M 16/026; A61B 5/08; A61B 5/4818; A61M 16/06; G06F 18/10; G06F 30/27; G06N 3/092; G16H 50/50; A61M 2016/0027

(57) ABSTRACT

Methods, devices and systems are described for adjusting airflow provided by a breathing assistance device to a user. In one aspect, personalized predictive models may be determined and used by the breathing assistance device to adjust the airflow to the user to improve various aspects including the user's sleep. In another aspect, models are developed for the user and the breathing assistance device to provide a digital twin to simulate the user and the device and determine if the user may develop any conditions and/or whether the device is operating properly. In another aspect, the personalized predictive model may be used in digital twin simulation results to validate performance before actual deployment.

### CROSS-REFERENCE TO RELATED APPLICATIONS

This application is a continuation of PCT/CA2024/050324, filed Mar. 18, 2024 (which designates the U.S.), which claims priority from U.S. Provisional Patent Application No. 63/453,093, filed on Mar. 18, 2023, the entire contents of which are hereby incorporated by reference in their entirety.

### FIELD

The various embodiments described herein generally relate to systems and methods for generating a personalized predictive model for management of airway pressure. Various embodiments relating to systems and methods for simulating the operation of a breathing assistance device and/or a health state of a user are also described herein.

### BACKGROUND

Individuals suffering acute or chronic respiratory (Chronic Obstructive Pulmonary Disease (COPD), asthma, Acute Respiratory Distress Syndrome (ARDS)) or respiratory-related conditions (e.g., sleep apnea) may require assistive devices to maintain respiratory functions at normal levels. Assistive devices such as mechanical ventilators, Positive Airway Pressure (PAP) devices or Continuous Positive Airway Pressure (CPAP) devices are common to provide breathing assistance. However, while such assistive devices are critical with respect to maintaining normal respiratory functions, these devices may also cause harm and distress to a user as a result of the stress or strain due to the amount of pressure or flow imparted on the user's respiratory system. Moreover, typically, most devices are currently reactive and not proactive to predict and prevent respiratory distress or discomfort. As such, there is a desire for methods and systems to identify and minimize user harm.

It is known in the art that there are various levels of mechanical support for different sorts of respiratory failure. In the most basic form, the inspired concentration of oxygen may be increased to percentages above 21% which is the normal atmospheric content of oxygen. This helps a patient in need to satisfy the metabolic need of oxygen for their body.

The next higher level of ventilatory support addresses the problem of when the oxygen content of the inhaled gas mixture is not sufficient to keep the homeostasis of the patient's body. This means that also retention of CO2 is becoming a problem. For these sorts of respiratory failures, a more invasive way of ventilation including active elevation of the airway pressure above the atmospheric pressure is involved to eliminate CO2 as the end product of the metabolism of the body. As this involves a tightly fitting mask, there are limits to the pressure that can be applied to the system.

If elevation of inhaled oxygen and increase of airway pressure facilitated by the mask is no longer sufficient then so-called mechanical ventilation using an intratracheal tightly fitted tube and/or tracheostomy along with a ventilator is used for ventilation. The parameters that are controlled with a ventilator include the volume of each breath applied to the patient, the respiratory rate per minute which, when taken together, allow for the volume of ventilation of the patient to be controlled along certain time intervals, e.g., every minute. In addition, typically mechanical ventilation also controls for the fraction of inhaled oxygen from 21% to 100% in air and the inspiratory to expiratory ratio of the breathing cycle. If these measures are not sufficient to keep blood oxygen and CO2 levels within safe physiological limits then opposed end expiratory pressure (PEEP) and an I/E inspiratory to expiratory ratio is applied. As far as monitoring of ventilation is concerned there are a variety of methods known in the art that include end tidal CO2, inspired CO2, inspired O2, expired O2, blood gas analysis of arterial blood pressure/volume diagrams and volumetric measures of the inspired and expired volumes of ventilation in the patient.

In sleep apnea, the “gold standard” diagnostic test for Obstructive Sleep Apnea (OSA) is polysomnography (PSG), in which respiratory, cardiac, muscular, and neurological parameters are monitored during sleep. The monitoring of these various physiological and neurological parameters allow for the evaluation of oxygen saturation of the blood, pauses of ventilation, EEG activity for determination of sleep phase, and EMG for determination of spontaneous muscular activity.

### SUMMARY OF VARIOUS EMBODIMENTS

According to one broad aspect of the teachings herein, in at least one embodiment described herein there is a method for generating a personalized predictive model for adjusting an airflow provided by a breathing assistance device to a user, wherein the method comprises: deploying a trained predictive model on a processor of a breathing assistance device controller, the trained predictive model being able to generate, based on sensor data, a nowcast of the user's current breathing state and a forecast of the user's future breathing state within a predicted time period; receiving the sensor data measured from one or more sensors; operating the processor of the breathing assistance device controller to apply the trained predictive model to the sensor data to generate the nowcast and the forecast; identifying one or more false negative predictions when the sensor data corresponding to a current period indicates a breathing event and the nowcast and/or the forecast corresponding to the current period indicates a normal breathing state; receiving false negative data comprising, for each of the one or more false negative predictions, the nowcast, the forecast, and a portion of the sensor data extending from a first time point before an onset of the false negative prediction to a second time point after an offset of the false negative prediction; generating the personalized predictive model by re-training the trained predictive model using the false negative data so that the personalized predictive model is personalized to the user; and deploying the personalized predictive model on the processor of the breathing assistance device controller.

In at least one embodiment, generating the personalized predictive model occurs after a minimum number of false negative predictions are identified.

In at least one embodiment, the method further comprises generating simulated false negative data for re-training the trained predictive model when an insufficient number of false negative data occurs during use by: applying one or more signal processing techniques to the sensor data, the one or more signal processing techniques comprising: jittering, noise addition, magnitude scaling, magnitude warping, filtering, phase warping, phase scaling, or chunk truncating.

In at least one embodiment, the minimum number of false negative predictions is in a range of one to a total number of time points at which the nowcast and the forecast are generated during a monitoring time period in which the airflow is provided by the breathing assistance device to the user.

In at least one embodiment, generating the personalized predictive model occurs automatically at a predetermined frequency.

In at least one embodiment, the method further includes determining one or more of a sensitivity, a precision, an F1 score and/or an adjusted F1 score of the trained predictive model, wherein generating the personalized predictive model occurs based on one or more of the sensitivity, the precision, the F1 score and/or the adjusted F1 score.

In at least one embodiment, the first time point and the second time point are in a range of 20 seconds to 60 seconds.

In at least one embodiment, method further comprising pre-processing the false negative data using one or more of: normalization, sensor data averaging, principal component analysis, independent component analysis, down sampling, up sampling, frequency filtering, or manual inspection of the sensor data.

In at least one embodiment, re-training the trained predictive model includes one or more of: transfer learning, tuning one or more parameters of the trained predictive model, adding a layer to the trained predictive model, reinforcement learning, or any combination thereof.

In at least one embodiment, re-training the trained predictive model further uses the sensor data.

In at least one embodiment, the predictive model comprises one or more nodes and one or more layers, and the one or more parameters of the trained predictive model include that is tunable include a type of node, a selection of nodes, a node weight, a node activation, a node memory, a number of connections between nodes, an orientation of connections between nodes, an orientation of connections between layers, a type of layer, a number of layers, a connection between layers, a number of inputs, a number of outputs, or an operable combination thereof.

In at least one embodiment, operating the processor of the breathing assistance device controller to apply the trained predictive model comprises: generating the nowcast by determining, based on the sensor data, a first plurality of probabilities, each of the first plurality of probabilities corresponding to a respective current breathing state of the user; and generating the forecast by determining, based on the sensor data, a second plurality of probabilities, each of the second plurality of probabilities corresponding to a respective predicted future breathing state of the user.

In at least one embodiment, the respective current breathing state of the user and the respective predicted future breathing state of the user comprise components including normal breathing or one or more respiratory failure events.

In at least one embodiment, the one or more respiratory failure events comprise components including obstructive apnea, central apnea, central hypopnea, obstructive hypopnea, respiratory effort related arousal, an unclassified event or any operable combination thereof.

In at least one embodiment, the one or more respiratory failure events include respiratory effort related arousal including flow limitation, snoring, oxygen desaturation, fragmentation, heart rate abnormality, or any combination thereof.

In at least one embodiment, the nowcast corresponding to the current period indicates the breathing event based on a first comparison of one or more of the first plurality of probabilities to one or more first thresholds and the forecast corresponding to the current period indicates the normal breathing state based on a second comparison of one or more of the second plurality of probabilities to one or more second thresholds.

In at least one embodiment, the one or more first thresholds and/or the one or more second thresholds are personalized to the user and adjustable.

In at least one embodiment, the one or more first thresholds and/or the one or more second thresholds are updated in real time, in near-real-time, hourly, daily, weekly, and/or monthly.

In at least one embodiment, the one or more sensors comprise user sensors, environmental sensors, or device sensors.

In at least one embodiment, the predicted time period is in a range of 20 seconds to 60 seconds.

In at least one embodiment, the false negative data is received at a remote processor that is remote located from the breathing assistance device, the personalized predictive model is generated at the remote processor, and the generated personalized predictive model is transmitted for deployment by the processor of the breathing assistance device controller via a network connection between the processor of the breathing assistance device controller and the remote processor.

In at least one embodiment, the personalized predictive model determines one or more of a pressure increase rate, a pressure decrease rate, and/or a pressure amplitude for adjusting the airflow provided by the breathing assistance device to the user.

In another aspect, there is provided at least one embodiment of a controller for controlling the operation of a breathing assistance device that provides breathing assistance to a user, wherein the controller comprises: a memory unit that comprises software instructions and parameters for at least one trained predictive model, the trained predictive model able to generate, based on sensor data, a nowcast of the user's current breathing state and a forecast of the user's future breathing state within a predicted time period; and a processor that is electronically coupled to the memory unit, the processor being configured to generate a control signal for controlling the breathing assistance device for a current monitoring time period by: receiving the sensor data obtained by one or more sensors, the sensor data including measurements of at least one airflow parameter of the user's airflow during the current monitoring time period when the user is using the breathing assistance device; applying the trained predictive model to generate the nowcast and the forecast; identifying one or more false negative predictions when the sensor data corresponding to the current monitoring time period indicates a breathing event and the nowcast and/or the forecast corresponding to the current monitoring time period indicates a normal breathing state; extracting false negative data comprising, for each of the one or more false negative predictions, the nowcast, the forecast, and a portion of the sensor data extending from a first time point before an onset of the false negative prediction to a second time point after an offset of the false negative prediction; generating a personalized predictive model by re-training the trained predictive model using the false negative data, the personalized predictive model being personalized to the user; and saving the personalized predictive model to the memory unit.

In at least one embodiment, the controller is further configured to perform the method as previously described.

In another aspect, there is provided a method for generating a personalized predictive model for adjusting an airflow provided by a breathing assistance device to a user, the method comprising: deploying a trained predictive model on a processor of a breathing assistance device controller, the trained predictive model being able to generate, based on sensor data, a nowcast of the user's current breathing state and a forecast of the user's future breathing state within a predicted time period; receiving the sensor data measured from one or more sensors; operating the processor of the breathing assistance device controller to apply the trained predictive model to generate the nowcast and the forecast; generating a summary representation of the user, the summary representation comprising user data; generating the personalized predictive model by conditioning the trained predictive model using the summary representation, the personalized predictive model being personalized to the user; and deploying the personalized predictive model on the processor of the breathing assistance device controller.

In at least one embodiment, the user data comprises one or more of the user's weight, height, gender, sex, age, body mass index, apnea-hypopnea index, SpO2, mask type of the breathing assistance device, prescribed pressure to be provided by the breathing assistance device, location type, or location elevation.

In at least one embodiment, the user data comprises one or more statistical representations of the user's breathing based on the sensor data, the one or more statistical representations of the user's breathing comprising an average waveform of a breath of the user, a variance for each sample timepoint within the average waveform, or one or more of a minimum, maximum, average, median, or variance of one or more of the user's air flow, air pressure, tidal volume, respiratory rate, SpO2, heart rate, sound, or motion.

In at least one embodiment, the user data comprises one or more statistical representations of the user's environment based on the sensor data, the one or more statistical representations of the user's environment comprising one or more of a minimum, maximum, average, median, or variance of one or more of temperature, ambient CO2, or ambient O2.

In at least one embodiment, the summary representation is generated by the trained predictive model using one or more embedding layers.

In at least one embodiment, conditioning the trained predictive model using the summary representation comprises: providing the summary representation as input to the trained predictive model; adapting a feature representation based on cross-attention with the summary representation, the feature representation being based on the sensor data; or providing the summary representation as input to a normalization block characterized by an offset factor and a scale factor for each feature corresponding to the normalization block being determined by a machine learning model conditioned on the summary representation.

In another aspect, there is provided a controller for controlling the operation of a breathing assistance device that provides breathing assistance to a user, wherein the controller comprises: a memory unit that comprises software instructions and parameters for at least one trained predictive model, the trained predictive model able to generate, based on sensor data, a nowcast of the user's current breathing state and a forecast of the user's future breathing state within a predicted time period; and a processor that is electronically coupled to the memory unit, the processor being configured to generate a control signal for controlling the breathing assistance device for a current monitoring time period by: receiving the sensor data obtained by one or more sensors, the sensor data corresponding to measurements of at least one airflow parameter of the user's airflow during the current monitoring time period when the user is using the breathing assistance device; applying the trained predictive model to generate the nowcast and the forecast; generating a summary representation of the user, the summary representation comprising user data; generating the personalized predictive model by conditioning the trained predictive model using the summary representation, the personalized predictive model being personalized to the user; and deploying the personalized predictive model on the processor of the breathing assistance device controller.

In at least one embodiment, the processor is further configured to perform the method as previously discussed.

In another aspect, there is provided a method for simulating one or more of an operation of a breathing assistance device and a health state of a user receiving assistance from the breathing assistance device, the method comprising: receiving a user model of the user, the user model being adapted for modeling one or more physiological systems of the user; receiving a device model of the breathing assistance device, the device model being adapted for modeling one or more components of the breathing assistance device, one or more subsystems of the breathing assistance device, or one or more functions of the breathing assistance device, or any operable combination thereof; receiving sensor data from one or more sensors; and determining an expected state of the breathing assistance device and/or an expected health state of the user based on applying the sensor data to the device model and/or the user model.

In at least one embodiment, the one or more sensors include any combination of: (a) one or more of user sensors placed on the user, (b) device sensors measuring properties of the breathing assistance device and (c) environmental sensors.

In at least one embodiment, the user sensors include a device for measuring blood parameters of the user.

In at least one embodiment, the user model is generated in part based on one or more personal characteristics of the user, the one or more personal characteristics of the user received from one or more of: the user, a medical professional, a pharmacy system, a payor system, a health system, other home medical equipment and an electronic medical records system.

In at least one embodiment, the one or more personal characteristics comprise a user alcohol consumption, a user drug consumption, medication taken by the user, a user height, a user weight, a user age, a blood test result, or any operable combination thereof.

In at least one embodiment, the user model is generated based on a model of the respiratory system of the user, a model of the cardiovascular system of the user a model of the nervous system of the user, or any operable combination thereof.

In at least one embodiment, the method further comprises: determining a current health state of the user by applying the sensor data to the user model; comparing the current health state of the user and the expected health state of the user; and generating a recommendation based on the comparison.

In at least one embodiment, the user model is associated with a physical model modeling one or more physiological systems of the user and determining the current health state of the user comprises applying the sensor data to the physical model of the user.

In at least one embodiment, the health state of the user comprises sleep health and wherein the method further comprises: determining one or more expected sleep parameter values for the user, by performing simulation by applying the sensor data to the user model and optionally the device; determining one or more actual sleep parameter values for the user based on received sensor data; comparing the one or more expected sleep parameter values and the one or more actual sleep parameter values; and generating a recommendation based on the comparison.

In at least one embodiment, the one more sleep parameter values comprise a duration of sleep, a length of stages of sleep, a depth of sleep, a sleeping heart rate or any operable combination thereof.

In at least one embodiment, the recommendation is a health recommendation, a recommendation to consult a medical professional, a diagnosis, a change of mask, a change of tubing, and/or an adjustment to the operation of the breathing assistance device.

In at least one embodiment, the expected state is a future state.

In at least one embodiment, the method further comprises: determining a device correction factor for the breathing assistance device for improving the health state of the user, where the determination is based on the expected health state of the user; simulating an operation of the breathing assistance device when the device correction factor is applied using the device model; and adjusting the operation of the breathing assistance device according to the correction factor when the simulation indicates an improvement in the heath state of the user when the correction factor is applied.

In at least one embodiment, wherein the correction factor is based in part on the sensor data received.

In at least one embodiment, the method further comprises: subsequent to adjusting the operation of the breathing assistance device, determining an intervention index characterizing a probability of the user experiencing a respiratory event in a given hour; and when the intervention index exceeds a predetermined intervention index threshold, re-adjusting the operation of the breathing assistance device by one of: reverting the operation of the breathing assistance device to an operation prior to the adjusting of the operation of the breathing assistance device or modifying the operation of the breathing assistance device to provide reactive therapy, wherein the probability of the user experiencing the respiratory event in the given hour is determined based on the user model.

In at least one embodiment, the method further comprises: subsequent to adjusting the operation of the breathing assistance device, determining a respiratory event index characterizing a number of respiratory events experienced by the user in one hour; and determining a mean historic number of respiratory events per hour experienced by the user; and when the respiratory event index exceeds the mean historic number of respiratory events by a predetermined respiratory event index threshold, re-adjusting the operation of the breathing assistance device by one of: reverting the operation of the breathing assistance device to an operation prior to the adjusting of the operation of the breathing assistance device or modifying the operation of the breathing assistance device to provide reactive therapy.

In at least one embodiment, adjusting the operation of the breathing assistance device comprises adjusting a mode of operation of the breathing assistance device, wherein the mode of operation is adjusted by selecting a device profile from: a continuous positive airway pressure (CPAP), an automatic positive airway pressure (APAP) profile, a bilevel positive airway pressure (BiPAP) profile, an adaptive servo-ventilation (ASV) profile and a non-invasive ventilator (NIV) profile.

In at least one embodiment, the method further comprises: determining a current state of the breathing assistance device based on the sensor data; comparing the current state of the breathing assistance device and an expected state of the breathing assistance device; in response to identifying a difference in the current state relative to the expected state, determining that one or more components of the breathing assistance device are malfunctioning; and in response to determining that the one or more components are malfunctioning, generating a device recommendation.

In at least one embodiment, the device recommendation is replacing the one or more components or performing maintenance on the one or more components.

In at least one embodiment, when the expected state of the breathing assistance device and/or the expected health state of the user is acceptable the method comprises deploying at least one model, calibration data, and/or an operational setting of the breathing assistance device that was used during the simulation to update future operation of the breathing assistance device.

In at least one embodiment, the received user model used during simulation also includes a personalized predictive model for the user, and the deployment includes sending the personalized predictive model for updating the future operation of the breathing assistance device.

In at least one embodiment, the method further comprises performing authentication by receiving a breathing signature for the user model and/or the sensor data, obtaining a stored breathing signature for the user, and performing the simulation when the received breathing signature for the user is the same as the stored breathing signature for the user.

In at least one embodiment, the method comprises provides an output for halting or adjusting therapy under situations where the simulation determines that an amount of air pressure is not safe for a user who has undergone surgery.

In at least one embodiment, the method comprises obtaining an increasing an amount of data logging and analyzing increased data to investigate whether sleep therapy has caused and/or aggravated a medical condition for the user.

In another aspect, there is provided a system for simulating one or more of an operation of a breathing assistance device and a health state of a user receiving assistance from the breathing assistance device, the system comprising: one or more sensors for measuring sensor data; a database storing a user model of the user, the user model being adapted for modeling one or more internal systems of the user, and a device model of the breathing assistance device, the device model being adapted for adapted for modeling one or more components of the breathing assistance device, one or more subsystems of the breathing assistance device, or one or more functions of the breathing assistance device, or any operable combination thereof; a controller in communication with the one or more sensors and the database, the controller comprising at least one processor configured to: receive the sensor data from one or more sensors; and determine one or more of an expected state of the breathing assistance device and an expected health state of the user based on one or more of the model of the breathing assistance device and the model of the user, and the sensor data.

In at least one embodiment, the processor is further configured to perform the method as previously described.

In another aspect, in accordance with the teachings herein, there is provided at least one embodiment of a method for adjusting an airflow provided by a breathing assistance device to a user, wherein the method comprises: determining a personalized predictive model for the user, the personal predictive model being adapted to control operation of the breathing assistance device specific to the user; performing simulation of the user and the breathing assistance device when the breathing assistance device is controlled by the personal predictive model; determining whether the simulation indicates that use of the personalized predictive model has a beneficial effect on health of the user; and when there is a beneficial effect on the health of the user, deploying the personalized predictive model to adjust future operation of the breathing assistance device.

In at least one embodiment, the personalized predictive model may be determined according to the teachings herein.

In at least one embodiment, the simulation is performed according to the teachings herein.

In at least one embodiment, false negative data associated with the personalized predictive model is used during the simulation.

In another aspect, in accordance with the teachings herein, there is provided at least one embodiment of a system for adjusting an airflow provided by a breathing assistance device to a user, wherein the system comprises a memory storing program instructions for a method for determining a personalized predictive model and a performing a digital simulation and a processor that is coupled to the memory to receive the program instructions, the processor being configured, when executing the program instructions, to perform the method previously described.

In another aspect, in accordance with the teachings herein, there is provided in at least one embodiment a non-transitory computer readable medium storing thereon program instructions that are executable by a processor for performing a method that is defined according in accordance with the teachings herein.

It will be appreciated that the foregoing summary sets out representative aspects of embodiments to assist skilled readers in understanding the following detailed description. Other features and advantages of the present application will become apparent from the following detailed description taken together with the accompanying drawings. It should be understood, however, that the detailed description and the specific examples, while indicating preferred embodiments of the application, are given by way of illustration only, since various changes and modifications within the spirit and scope of the application will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1A is a block diagram of an example embodiment of a breathing assistance system for controlling or tuning a breathing assistance device during use by a user based on determining and/or predicting different sleep and/or respiratory behaviors in accordance with the teachings herein.

FIG. 1B is a schematic diagram of another example embodiment of a breathing assistance system for controlling or tuning a breathing assistance device during use by a user based on determining and/or predicting different sleep and/or respiratory behaviors in accordance with the teachings herein.

FIG. 2 is a block diagram of another example embodiment of a breathing assistance system for controlling or tuning a breathing assistance device during use by a user based on determining and/or predicting different sleep and/or respiratory behaviors in accordance with the teachings herein.

FIG. 3A is a flowchart of an example embodiment of a method for generating a personalized predictive model that can be used to adjust an airflow provided by a breathing assistance device to a user in accordance with the teachings herein.

FIG. 3B is an example flowchart of the flow of data through an example breathing assistance system in accordance with the teachings herein.

FIG. 4A shows example sensor data comprising pressure and airflow data.

FIG. 4B shows example nowcast and forecast data generated by an example trained predictive model in accordance with the teachings herein.

FIG. 4C shows example nowcast and forecast data generated by an example personalized predictive model in accordance with the teachings herein.

FIG. 4D shows example sensor data comprising pressure and airflow data and example forecast data generated by an example personalized predictive model in accordance with the teachings herein.

FIG. 5A shows example sensor data comprising airflow data, example forecast data generated by an example general trained predictive model, and example forecast data generated by an example personalized predictive model in accordance with the teachings herein.

FIG. 5B shows further example sensor data comprising airflow data, example forecast data generated by an example general trained predictive model, and example forecast data generated by an example personalized predictive model in accordance with the teachings herein.

FIG. 5C shows further still example sensor data comprising airflow data, example forecast data generated by an example general trained predictive model, and example forecast data generated by an example personalized predictive model in accordance with the teachings herein.

FIG. 6A shows example sensitivity data corresponding to an example general trained predictive model and an example personalized predictive model in accordance with the teachings herein.

FIG. 6B shows example precision data corresponding to an example general trained predictive model and an example personalized predictive model in accordance with the teachings herein.

FIG. 7 is a flowchart of an example embodiment of a method for simulating an operation of a breathing assistance device and/or of a health state of a user receiving assistance from the breathing assistance device.

Further aspects and features of the example embodiments described herein will appear from the following description taken together with the accompanying drawings.

### DETAILED DESCRIPTION OF THE EMBODIMENTS

The headings and Abstract of the Disclosure provided herein are for convenience only and do not interpret the scope or meaning of the embodiments.

Various embodiments in accordance with the teachings herein will be described below to provide examples of at least one embodiment of the claimed subject matter. No embodiment described herein limits any claimed subject matter. The claimed subject matter is not limited to devices, systems or methods having all of the features of any one of the devices, systems or methods described below or to features common to multiple or all of the devices, systems or methods described herein. It is possible that there may be a device, system or method described herein that is not an embodiment of any claimed subject matter. Any subject matter that is described herein that is not claimed in this document may be the subject matter of another protective instrument, for example, a continuing patent application, and the applicants, inventors or owners do not intend to abandon, disclaim or dedicate to the public any such subject matter by its disclosure in this document.

Furthermore, it will be appreciated that for simplicity and clarity of illustration, where considered appropriate, reference numerals may be repeated among the figures to indicate corresponding or analogous elements or steps. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

It should also be noted that the terms “coupled” or “coupling” as used herein can have several different meanings depending in the context in which these terms are used. For example, the terms coupled or coupling can have a mechanical, electrical or communicative connotation. For example, as used herein, the terms coupled or coupling can indicate that two elements or devices can be directly connected to one another or connected to one another through one or more intermediate elements or devices via an electrical element, electrical signal or a mechanical element depending on the particular context.

Unless the context requires otherwise, throughout the specification and claims which follow, the word “comprise” and variations thereof, such as, “comprises” and “comprising” are to be construed in an open, inclusive sense, that is, as “including, but not limited to”.

Various terms used throughout the present description may be read and understood as follows, unless the context indicates otherwise: singular articles and pronouns as used throughout include their plural forms, and vice versa; similarly, gendered pronouns include their counterpart pronouns so that pronouns should not be understood as limiting anything described herein to use, implementation, performance, etc. by a single gender. Further definitions for terms may be set out herein; these may apply to prior and subsequent instances of those terms, as will be understood from a reading of the present description.

It should also be noted that, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof. As another example, the phrase “A, B, C or any operable combination thereof” or “any combination of A, B and C” is mean to cover any combination of elements A, B and C that provides utility which may, for example, include A, B, C, A and B, A and C, B and C, or A, B and C.

It should be noted that terms of degree such as “substantially”, “about” and “approximately” as used herein mean a reasonable amount of deviation of the modified term such that the end result is not significantly changed. These terms of degree may also be construed as including a deviation of the modified term, such as by 1%, 2%, 5% or 10%, for example, if this deviation does not negate the meaning of the term it modifies.

Furthermore, the recitation of numerical ranges by endpoints herein includes all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed, such as 1%, 2%, 5%, or 10%, for example.

Reference throughout this specification to “one embodiment”, “an embodiment”, “at least one embodiment” or “some embodiments” means that one or more particular features, structures, or characteristics may be combined in any suitable manner in one or more embodiments, unless otherwise specified to be not combinable or to be alternative options.

As used in this specification and the appended claims, the singular forms “a,” “an,” and “the” include plural referents unless the content clearly dictates otherwise. It should also be noted that the term “or” is generally employed in its broadest sense, that is, as meaning “and/or” unless the content clearly dictates otherwise.

Similarly, throughout this specification and the appended claims the term “communicative” as in “communicative pathway,” “communicative coupling,” and in variants such as “communicatively coupled,” is generally used to refer to any engineered arrangement for transferring and/or exchanging information. Examples of communicative pathways include, but are not limited to, electrically conductive pathways (e.g., electrically conductive wires, physiological signal conduction), electromagnetically radiative pathways (e.g., radio waves), or any combination thereof. Examples of communicative couplings include, but are not limited to, electrical couplings, magnetic couplings, radio couplings, or any combination thereof.

Throughout this specification and the appended claims, infinitive verb forms are often used. Examples include, without limitation: “to detect,” “to provide,” “to transmit,” “to communicate,” “to process,” “to route,” and the like. Unless the specific context requires otherwise, such infinitive verb forms are used in an open, inclusive sense, that is as “to, at least, detect,” to, at least, provide,” “to, at least, transmit,” and so on.

A portion of the example embodiments of the systems, devices, or methods described in accordance with the teachings herein may be implemented as a combination of hardware or software. For example, a portion of the embodiments described herein may be implemented, at least in part, by using one or more computer programs, executing on one or more programmable devices comprising at least one processing element, and at least one data storage element (including volatile and non-volatile memory). These devices may also have at least one input device (e.g., a keyboard, a mouse, a touchscreen, and the like) and at least one output device (e.g., a display screen, a printer, a wireless radio, and the like) depending on the nature of the device.

It should also be noted that there may be some elements that are used to implement at least part of the embodiments described herein that may be implemented via software that is written in a high-level procedural language such as object-oriented programming. The program code may be written in C, C⁺⁺ or any other suitable programming language and may comprise modules or classes, as is known to those skilled in object-oriented programming. Alternatively, or in addition thereto, some of these elements implemented via software may be written in assembly language, machine language, or firmware as needed.

At least some of the software programs used to implement at least one of the embodiments described herein may be stored on a storage media or a device that is readable by a general or special purpose programmable device. The software program code, when read by the programmable device, configures the programmable device to operate in a new, specific and predefined manner in order to perform at least one of the methods described herein.

Furthermore, at least some of the programs associated with the systems and methods of the embodiments described herein may be capable of being distributed in a computer program product comprising a computer readable medium that bears computer usable instructions, such as program code, for one or more processors. The program code may be preinstalled and embedded during manufacture and/or may be later installed as an update for an already deployed computing system. The medium may be provided in various forms, including non-transitory forms such as, but not limited to, one or more diskettes, compact disks, tapes, chips, and magnetic and electronic storage. In alternative embodiments, the medium may be transitory in nature such as, but not limited to, wire-line transmissions, satellite transmissions, internet transmissions (e.g., downloads), media, digital and analog signals, and the like. The computer useable instructions may also be in various formats, including compiled and non-compiled code.

Accordingly, any module, unit, component, server, computer, terminal or device described herein that executes software instructions may include or otherwise have access to computer readable media such as storage media, computer storage media, or data storage devices (removable and/or non-removable) such as, for example, magnetic disks, optical disks, or tape. Computer storage media may include volatile and non-volatile, removable and non-removable media implemented in any method or technology for storage of information, such as computer readable instructions, data structures, program modules, or other data. Examples of computer storage media include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information, and which can be accessed by an application, module, or both. Any such computer storage media may be part of the device or accessible or connectable thereto.

It should be noted that the term “user” covers a person who is using a breathing assistance device. In some cases, the user may be an individual that is using the breathing assistance device in their home or a non-medical setting. In other cases, the user may be a patient who is using the breathing assistance device in a medical setting such as a clinic or a hospital, for example.

A respiratory failure may be understood to cover all diseases and conditions which can result in a negative change in a person or animal's respiratory system such as a breathing obstruction or small airways. In some cases, the respiratory failure may be a temporary respiratory event that occurs such as during OSA or an asthma attack or it may be due to a chronic respiratory condition such as lung cancer, cystic fibrosis or chronic obstructive pulmonary disease.

In accordance with the teachings herein, there is provided various embodiments for intelligently monitoring, analyzing, determining and/or predicting different respiratory and sleep behaviors of a user, such as generating a personalized predictive model that is specific to a particular user to detect and/or predict the user's breathing state and using a digital model of the user (e.g., digital twin) and/or a digital model of a breathing assistance device providing assistance to the user to simulate a sleep health of the user, which may include predicting a future sleep health of the user. The various embodiments can be used to adjust the operation of the breathing assistance device. Various embodiments for monitoring the operation of a breathing assistance and predicting a future operation of the breathing assistance device by simulating the operation of the breathing assistance device using a digital model of the breathing assistance model are also provided.

At least some of the methods involve using machine learning and other advanced computational techniques to build accurate supervised classification and prediction models. Some of these models will employ various measurements, some of which include, but are not limited to: (a) air pressure and air flow only; (b) FOT measured airway impedance only; (c) plethysmography such as EEG, (d) other sensor data as described herein or (e) a combination of two or more of (a), (b), (c), and other sensor data described herein.

In one aspect, the teachings herein may use data associated with the forced oscillation technique in the detection and/or prediction of respiratory failure which may be performed in various ways such as, but not limited to, the techniques described in U.S. Pat. No. 11,633,560 entitled “METHOD AND APPARATUS FOR CONTINUOUS MANAGEMENT OF AIRWAY PRESSURE FOR DETECTION AND/OR PREDICTION OF RESPIRATORY FAILURE”, and/or U.S. Pat. No. 11,612,708 entitled “METHOD & APPARATUS FOR DETERMINING AND/OR PREDICTING SLEEP AND RESPIRATORY BEHAVIOURS FOR MANAGEMENT OF AIRWAY PRESSURE”, which are each hereby incorporated by reference in their entirety.

In one aspect, in at least one embodiment, the teachings herein provide techniques for generating a personalized predictive model that can be based, in some cases, on physiological measurements of the user, or values calculated from such physiological measurements, such as a user's breathing signature, classification of the user's sleep stage and/or classification of predicted sleep disruption severity. Based on the detection and/or prediction indicated by the personalized predictive model, the operating parameters of the breathing assistance device may then be adjusted to improve the chances that the user of the breathing assistance device does not experience a respiratory failure event or at least experiences minimal respiratory failure events. Accordingly, at least one of these detection and/or prediction indications can be used to generate a feedback control signal that is used to control the operation of the breathing assistance device. These indications may be determined for a given time period and can be used to control the breathing assistance device over the given time period.

Therefore, in one aspect, in at least one embodiment, the teachings herein provide for real time personalized detection and/or prediction of the user's breathing state based on a personalized predictive model, which may then be used to perform at least one of adjusting the operation of the breathing assistive device and providing data in a user report that can be used to monitor the breathing of the user and/or diagnose a respiratory disorder for the user. For example, the report can be determined for data collected when the user slept at night and the report can be provided to the user or a medical professional for review, such as in the morning, so that the user or the medical professional can review data about their respiratory and sleep health, for example. Various example embodiments of the personalized predictive model are provided herein. The predictive model may employ machine learning models also referred to as AI models.

Previously it was not possible to predict an upcoming respiratory failure event for the user of a breathing assistance device in an automated fashion before the respiratory failure was about to occur. Accordingly, breathing assistance devices were conventionally controlled in a manual fashion by a medical practitioner who set and then adjusted the operational parameters of the breathing assistance device every so often. This was detrimental since if the user started experiencing respiratory failure it was not conventionally possible to automatically adjust the breathing assistance device to reduce the effect or amount of respiratory failure encountered by the user which may be fatal in some situations where response time is critical for adjusting the operation of the breathing assistance device. Furthermore, such conventional techniques will not even allow for the prediction of imminent respiratory failure.

More recently, other techniques including traditional FOT/Oscillometry have been used to automatically adjust the parameters of breathing assistance devices. However, traditional FOT uses averaging and therefore there is a delay of multiple seconds before any detection can happen. This is also detrimental to the user health when a significant respiratory failure is imminent or is occurring. Moreover, automatic adjusting of breathing assistance devices utilizing techniques such as only sensing the airflow or oxygen levels may not provide enough information of the health of the complete respiratory system in certain situations.

It is believed that the techniques of generating a personalized predictive model for detecting and/or predicting a user's breathing state to generate a control signal to control the breathing assistance device to maintain the respiratory health of the user in a certain range where the user is not experiencing a respiratory failure event, in at least one embodiment provided in accordance with the teachings herein, will increase the rate of adoption of use of breathing assistance devices where the use is voluntary (i.e., as for sleep apnea devices). Such models also provide technical advantages such as an increase in the speed of adaptation of the breathing assistance device to any respiratory failure event that is currently being encountered or preferably the prevention of a respiratory failure event that may soon be encountered by the user as methods using personalized AI models described herein can detect and/or predict the respiratory failure relatively quickly, with increased confidence, and can also preferably take proactive or reactive steps quickly to control the breathing assistance device to reduce the level/amount of respiratory failure that is encountered by the user or prevent the respiratory failure event from even happening. This can be critical in some cases where increased respiratory failure events can have significant, if not fatal, consequences to the user.

In another aspect, the teachings herein provide at least one embodiment for simulating an operation of a breathing assistance device and/or simulating a health state of a user, for example, a user receiving assistance from the breathing assistance device, using various models of the user (i.e., user model) and/or of the breathing assistance device (i.e., device model). Simulating a health state of a user using a user model through the use of a “digital twin” of the user, which can be used to evaluate a current health state of the user and compare the current health state with an expected health state to assess the therapy provided by the breathing assistance device, and/or to predict a future condition of the user if the therapy was to continue, such as a future user health state, a future user sleep health and/or future user breathing health and/or a future device operation of the breathing assistance device. By predicting health events and/or health conditions or diseases, it may be possible to avoid or lower the likelihood of the events and/or conditions or diseases occurring or seek early treatment. In some cases, the prediction(s) can be used to make recommendations and/or to modify the operation of the breathing assistance device to adjust the therapy provided to the user such as, for example, to improve the therapy provided to the user. In at least one example embodiment, the device model of the breathing assistance device and/or of the user along with (a) data from the breathing assistance device, (b) sensor data from sensors on the user and/or in the user's room, (c) user data provided by the user and/or by a medical professional supervising the user's therapy and/or (d) test results may be used to simulate the effects of changes to the therapy provided by the breathing assistance device, which may be helpful to identify optimal or improved adjustments to the operation of the breathing assistance device. For example, (i) adjustments may be made to the operation of the breathing assistance device, (ii) recommendations may be provided to the patient to adjust their mask, change the tube used with the breathing assistance device, change the user's mask type, and/or change other sleeping set up specific to the user (iii) reports and/or alerts may be sent to the patient and/or to an external user such as a medical professional overseeing the patient's therapy or (iv) a combination of two or more of (i), (ii) and (iii). As another example, simulating the operation of the breathing assistance device through the use a “digital twin” of the breathing assistance device can help identify malfunctioning components of the breathing assistance device or make predictions about future fault conditions that may be experienced by the breathing assistance device.

The various personalized models, user models and device models described herein may be developed using a combination of models, for example, analog models, digital models, mathematical models, neural network models and/or other machine learning models, for modeling user physiological systems which may affect the state of health of a user, including a sleep health, a cardiovascular health, a respiratory health and/or brain health of the user, and for modeling the user's environment and/or environmental factors that may affect the state of health of the user. The models may be personalized by adjusting parameters of the models so that the models can more accurately reflect the health state of a given user from the health of another user and/or reflect a current environment of the user. The breathing assistance device model may similarly be developed using a combination of models to model the operation and/or the components of the breathing assistance device.

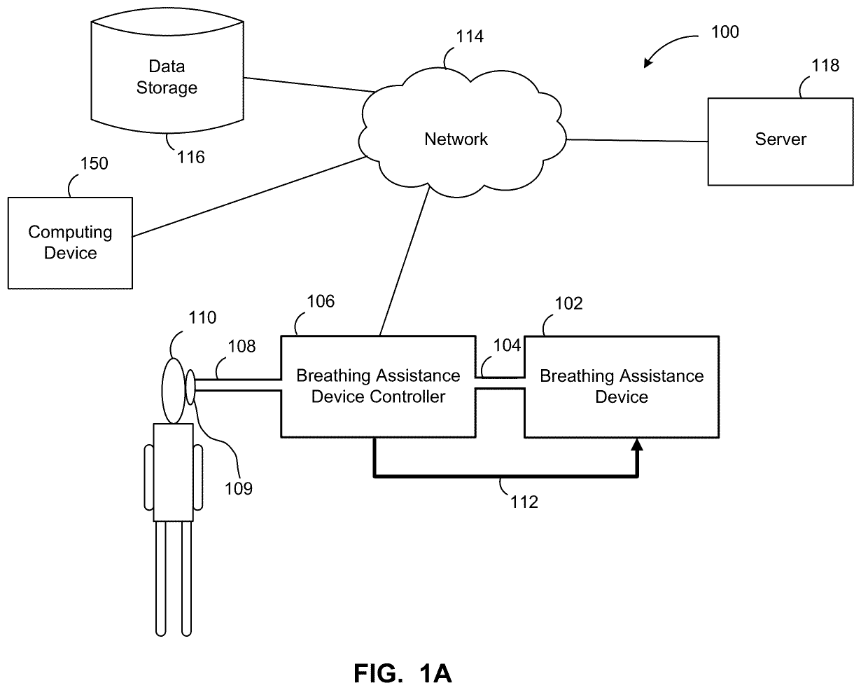

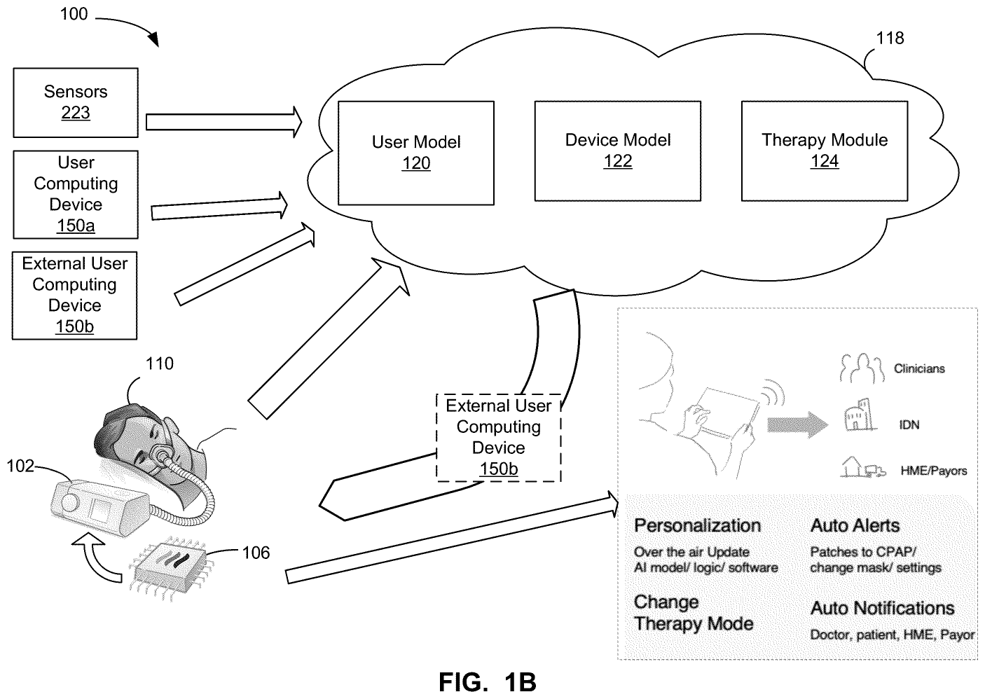

Referring now to FIG. 1A, illustrated therein is a block diagram of a breathing assistance system 100 that may use a personalized predictive model for adjusting an airflow provided by a breathing assistance device to a user and/or to simulate an operation of the breathing assistance device and/or a health state of the user receiving assistance form the breathing assistance device in communication with external components via network 114. In at least one embodiment, the breathing assistance system 100 can also be used for controlling or tuning the air pressure or an airflow provided by a breathing assistance device to the user using the forced oscillation technique based on detection and/or prediction of a respiratory failure event using one or more personalized models in accordance with at least one embodiment of the teachings herein. The system 100 comprises a breathing assistance device 102 that generates an airflow that is provided to a user 110 via air transport pathways 104 and 108 and, for example, a laryngeal tube, a breathing mask or an endotracheal tube 109 (hereinafter collectively referred to as an “entry element”). The airflow can be at least one pressure pulse of air, a continuous flow of air, or another type of airflow as is known by those skilled in the art. The airflow is controllable by adjusting at least one of the air pressure and flow rate of the breathing assistance device 102 via corresponding input controls on the breathing assistance device 102.

In some embodiments, the breathing assistance device 102 may be a mechanical ventilator for providing breathing support to the user. In other embodiments, the breathing assistance device 102 may be a CPAP, APAP, BiPAP, PAP, ASV, NIV, or High Flow Oxygen (HFO) device for providing breathing support to the user. In other embodiments, the breathing assistance device 102 may be a respiratory treatment delivery device such as, but not limited to, respiratory treatment delivery devices that assist a user in clearing their lungs and coughing out secretions. In other instances, the breathing assistance device 102 may be an anesthesia machine in the OR, an ICU ventilator, a home ventilator and oxygenator of COPD, and any other machine that provides breathing assistance to a user who has a respiratory disease. Therefore, in general, the teachings described herein for the detection and/or prediction of a respiratory failure event and the proactive or reactive actions that are taken to reduce, remove or pre-empt respiratory failure can be used with all types of ventilation including invasive (with tube) and non-invasive (tubeless) ventilation.

A respiratory failure event can include any event that deviates from normal breathing. For example, in sleep apnea related breathing, a respiratory failure event may include a flow limitation, snoring, obstructive apnea, central apnea, obstructive hypopnea, central hypopnea, respiratory effort related arousals (RERA), and/or arousals. For example, respiratory effort related arousals may include flow limitation, snoring, oxygen desaturation, fragmentation, heart rate abnormality, or any combination thereof. As another example, in users with diseases such as chronic obstructive pulmonary disease, a respiratory failure event may include stiffening of the lung, exacerbation, and/or flow limitation. As another example, in users using an ICU ventilator, a respiratory failure event may include failing of weaning of the user from ventilation, pneumonia, and/or resistive or elastic events of the lung. Other respiratory failure events may be determined.

A breathing assistance device controller 106 is coupled to the breathing assistance device 102 via the air transport pathway 104 (which may also be called the flow passage 104) and receives airflow from the breathing assistance device 102 and delivers the airflow via the air transport pathway 108 and the entry element 109 to the user 110. It should be noted that the term “air” in the present disclosure is used generally to denote the flow of gas and other airborne particles through the system 100. For example, the output of a mechanical ventilator may include gasses and/or vapors other than air such as, but not limited to, anesthetics, for example which are typically vapors but can also be gases. In a PAP device, water vapor may be combined with air. In some embodiments of the breathing assistance device 102, gaseous medication (i.e., steroids, oxygen, Nitrogen, etc.) may be added to the air flow and provided to the patient under ventilation based on respiratory health and/or measured comfort level. For example, the medication may include an appropriate amount of steroids that may be used daily to improve the CPAP experience for the user. The airflow may be delivered to the user 110 via the entry element 109. In the present embodiment, the entry element 109 may be a mask worn over the user's 110 nose and mouth, just over the nose or adjacent or inside the nostrils of the user 110 for alternative masks. In other embodiments, the entry element 109 may be an endotracheal tube inserted into the trachea by means of intubation or tracheostomy.

In embodiments in which the breathing device 102 is a mechanical ventilator, there are actually two air pathways (not shown) instead of just the air transport pathway 104 (which may also be called a flow passage) where one of the pathways is used for inhalation and the other of these pathways is used for exhalation. The pathways shown in FIG. 1 apply for the case where the breathing assistance device 102 is a PAP device. It may be thus understood that the breathing assistance device 102 provides at least one pathway to allow air to flow from the air transport pathway 104 to the air transport pathway 108. It may further be understood that there can be embodiments in which the breathing assistance device controller 106 is at least partially or completely incorporated “inline” with the airflow pathways from the breathing assistance device 102 to the user 110.

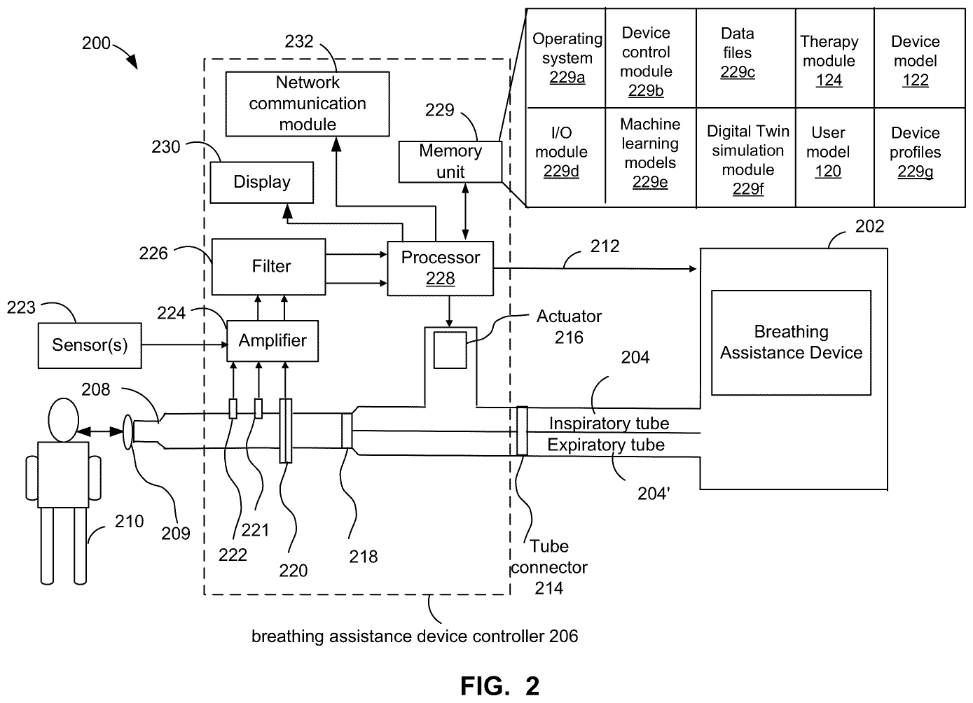

In the present example embodiment, the breathing assistance device controller 106 comprises one or more sensors (e.g., see FIG. 2) to measure various parameters of the airflow being delivered to the user 110. For example, sensors can be attached to the mask worn by the user 110 which may result in improved SNR for the sensor data obtained from the sensors. Alternatively, the sensors, such as ultrasonic sensors for example, can be attached in the tubing pathway. In either case, these sensors can be used to measure airflow parameters associated with both inspiration and expiration. However, in the case of a PAP machine, such sensors are located close to the mask because the tube 108 only carries an inspiratory flow whereas in a mechanical ventilator the sensors can be attached to the mask or endotracheal tube or they can be located anywhere along the tubes that are used for the inspiratory pathway and the expiratory pathway.

In some embodiments, the breathing assistance device controller 106 may not include these sensors but may instead read these parameters from the breathing assistance device 102 since the breathing assistance device 102 may also be equipped with sensors for measuring airflow parameters. In some embodiments, the breathing assistance device controller 106 transmits data including sensor data to an external system and/or receives data from an external system that is used for adjusting the operation of the breathing assistance device 102 (e.g., see FIG. 1B). The breathing assistance device controller 106 may further comprise a device to provide a forced oscillation signal, in order to provide changes in air pressure for the airflow provided to the user 110. In some embodiments, a sensor for measuring both air pressure and airflow is present. In other embodiments, dedicated sensors may be used to measure the airflow or the air pressure such that more than one sensor may be used with the breathing assistance device controller 106. For example, some sensor technologies use a laser to detect movement or ultrasound can be used to detect both pressure and flow rate using one sensor (as the measured flow rate can be determined from dividing the measured pressure by a known resistance).

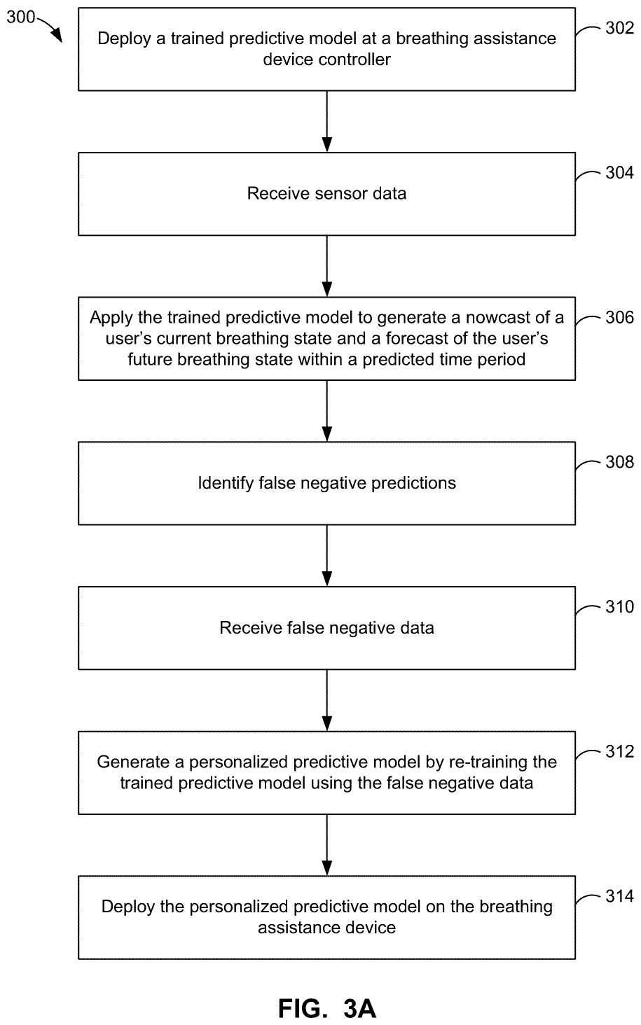

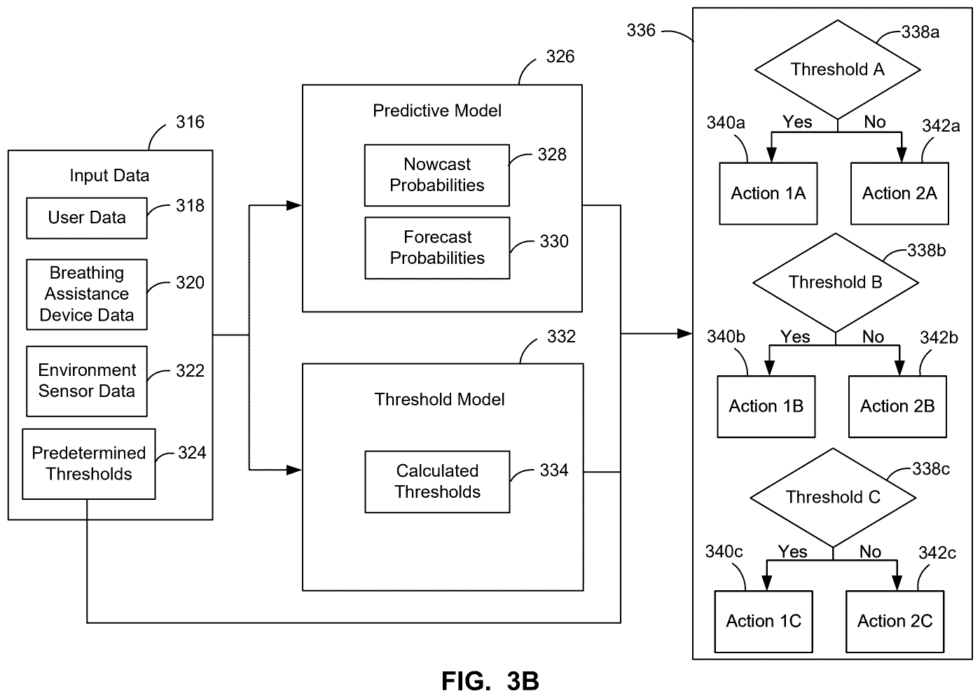

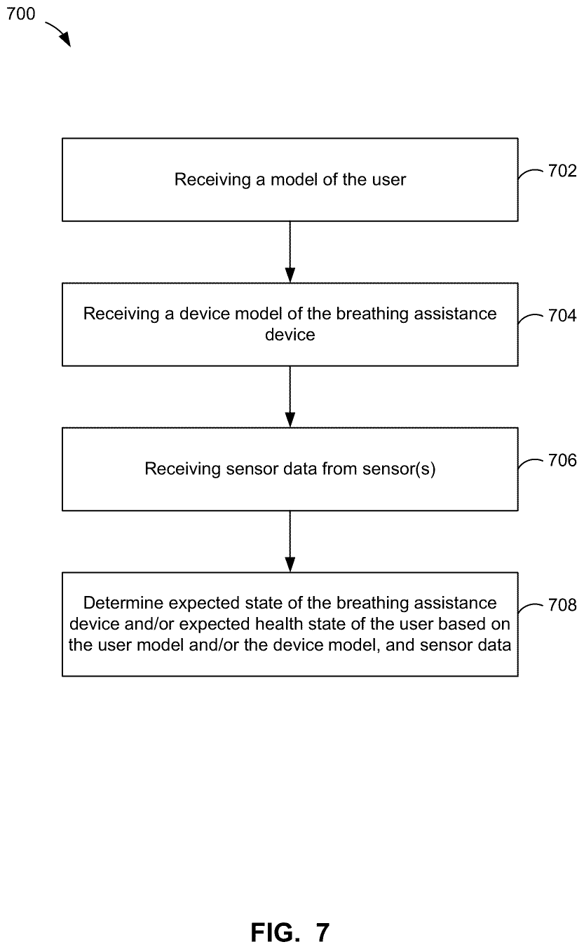

The measured airflow parameters such as air volume, air pressure and/or airflow rate may be used by the breathing assistance device controller 106 to generate a control signal 112 that can be used as feedback to adjust the operation of the breathing assistance device 102. For example, the breathing assistance device controller 106 can employ a control method, where the control signal that is generated may be based on a trained predictive model such as, but not limited to, a personalized predictive model (e.g., according to method 300 in FIG. 3A) and/or a current or predicted future sleep or respiratory health of the user and/or a current or predicted future operation of the breathing assistance device (e.g., according to method 700 in FIG. 7). The personalized predictive model can be trained using data specific to the user and the predicted future sleep and respiratory health of the user can be determined using model(s) specific to the user, and accordingly a control signal specific to the user can be generated.

For example, the breathing assistance controller 106 can employ a control method that uses various therapy models including a trained predictive model and/or a personalized predictive model, such as those described with reference to method 300 (see FIG. 3A), for example, to predict when a respiratory failure event will occur (e.g. perhaps up to and including the next few minutes such as for example from about a few milliseconds up to about 5 minutes) and to then generate the control signal to provide a proactive action so that the user does not experience the predicted respiratory failure event. As another example, the breathing assistance controller 106 can employ a control method that uses (a) a user digital model, (b) a breathing assistance device digital model, such as those described with reference to method 700 (see FIG. 7) to predict an expected state of the breathing assistance device and/or (c) an expected health state of the user and to then generate the control signal to adjust the operation of the breathing assistance device to improve the expected state of the breathing assistance device and/or of the user and provide pre-emptive therapy. In such embodiments, in at least one embodiment, additional measured signals can be used to implement the predictive method. For example, the additional measured signals can be one or more of the physiological or and/or neurological signals that are obtained during polysomnography (PSG) and hereafter referred to as PSG signals. The PSG signals may include at least one physiological signal (such as but not limited to eye movements (EOG), muscle activity or skeletal muscle activation (EMG) and cardiac signals (ECG)) and/or at least one neurological signal (such as but not limited to EEG). The PSG signals may further include air pressure, air flow, chest movement tracked by a belt worn around a user's chest, SPO2, pulse oximetry, audio signals, camera signals, temperature signals, heart rate or any operable combination thereof. In some embodiments, the additional measured signals may include other types of data such as those measured by sound sensors (e.g., microphones), movement detection and/or recording systems (e.g., image and video acquisition and/or recording systems, and/or radar and/or electromagnetic recording systems), humidity sensors, alcohol and/or substance sensors, position sensors (e.g., gyroscopes and/or accelerometers), light sensors, pressure sensors or any operable combination thereof. Sensors for measuring such signals are known by those skilled in the art and can be added into the system 100 depending on the particular embodiment that is used.

In various embodiments described herein, the control signal 112 may be used to adjust one, two, a few or all of the adjustable parameters of the breathing assistance device 102 to prevent or reduce the severity of a respiratory failure event. For example, parameters that may be adjusted include the flow rate of the airflow, the volume of the airflow, the pressure of the airflow, the pressure of airflow increase rate, the pressure of airflow decrease rate, the frequency of certain changes in the airflow (like changes in the flow rate, volume, pressure and amplitude of the airflow), the amplitude of the airflow and/or the phase of the airflow that can be generated by the breathing assistance device 102, or an operable combination thereof.

In the present example embodiment, the breathing assistance device controller 106 comprises a network communication module (not shown) that enables communication with a server 118 and a data storage 116 via a network 114. Although one server 118 is shown it should be understood that there may comprise one or more servers 118 that are distributed over a wide geographic area and may communicate via the network 114 with the breathing assistance device controller 106. For example, the server 118 can be configured to receive data from the breathing assistance device controller 106. The server 118 can further be configured to generate and/or update models, including a user model 120 of the user, a device model 122 of the breathing assistance device 102 and various therapy models implemented by the therapy module 124 as shown in FIG. 1B, including, predictive models. In at least one embodiment, the predictive model(s) comprise predictive model(s) trained using a pool of group data. In at least one embodiment, the predictive model comprises one or more personalized predictive models of the user and/or of the breathing assistance device that are trained and/or developed using data received from the breathing assistance device controller 106 and/or from sensors (not shown). In at least one embodiment, the server 118 is configured to generate the personalized predictive model(s) using one or more re-training techniques using the data received from the breathing assistance device controller 106. In at least one embodiment, the server 118 receives data from the data storage 116. For example, in at least one embodiment, the predictive model(s) are generated using group data received from the data storage 116. The server 118 may also be configured to save data, such as one or more models, to the data storage 116. The server 118 can be a cloud-based server, as shown in FIG. 1B.

The data storage 116 can include RAM, ROM, one or more hard drives, one or more flash drives or some other suitable data storage elements such as disk drives, etc. For example, the data storage 116 may include one or more databases or files (both not shown) for storing, for example, sensor data, predictive models, training data, testing data, and/or validation data. The data storage 116 can further be used to store information related to sensor data, such as, for example, user data with respect to the sensor data, predictive models, training data, testing data, and/or validation data. Sensor data, predictive models, training data, testing data, validation data, and/or information related to the sensor data stored in the data storage 116 can be retrieved by the breathing assistance device controller 106 and/or the server 118. The data storage 116 may also include database(s) for storing information related to the user that may be used to train and/or generate the predictive model(s) and information relating to results obtained from applying the model(s), for example recommendations and reports. In some embodiments, the data storage 116 is cloud-based.

The computing device 150 can be include any device capable of communicating with other devices through a network such as the network 114. A network device can couple to the network 114 through a wired or wireless connection. The computing device 106 can include a processor and memory, and may be an electronic tablet device, a personal computer, workstation, server, portable computer, mobile device, personal digital assistant, laptop, smart phone, WAP phone, an interactive television, video display terminals, gaming consoles, and portable electronic devices or any combination of these. The computing device 150 can be a device used for inputting and/or receiving data about the user and/or for receiving alerts and/or notifications associated with the therapy of the user. Although only one computing device 150 is shown, it will be understood that more than one computing device 150 can be in communication with the breathing assistance device 102, the data storage 116 and the server 118. For example, a first computing device 150 may be associated with a medical professional overseeing the user's 110 therapy and a second computing device 150 may be associated with the user 110.

The network 114 may be any network capable of carrying data, including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g., Wi-Fi, WiMAX), SS7 signaling network, fixed line, local area network, wide area network, and others, including any combination of these, capable of interfacing with, and enabling communication between, the breathing assistance device controller 106, the server 118, and the data storage 116.

Referring now to FIG. 2, shown therein is a block diagram of an example embodiment of a breathing assistance system 200 that may be used to generate personalized predictive model(s) of the user and/or simulate the operation of a breathing assistance device 202 and/or a health state of the user. The breathing assistance device 200 may be used to control or tune the breathing assistance device 202 during use by a user 210 based on detection and/or prediction of respiratory failure, analysis of simulations of the operation of breathing assistance device 202 and/or analysis of simulations of the health state of the user, in accordance with the teachings herein.

Elements in FIG. 2 that correspond to elements in FIG. 1A have been numbered similarly. Similar to the configuration of the breathing assistance system 100, a breathing assistance device 202 generates airflow that is provided to a user 210 via air transport pathways 204 and 204′ and the breathing tube 208 and the airflow is monitored by a breathing assistance device controller 206 for modifying the operation of the breathing assistance device 202 under certain conditions. Similar to FIG. 1A, the airflow may be delivered to the user 210 via an entry element 209. In this example embodiment the entry element 209 may be a mask worn over to fluidically couple with the user's 210 nose (i.e., nostrils) and optionally the user's mouth. In other embodiments, the entry element 209 may be an endotracheal tube inserted into the trachea by means of intubation or tracheostomy.

FIG. 2 provides additional details with respect to the various system components that may be employed. In some embodiments, the breathing assistance device 202 may be a mechanical ventilator for providing breathing support. In other embodiments, the breathing assistance device 202 may be a PAP device for providing breathing support. Other options are available for the breathing assistance device 202 as explained for the breathing assistance device 102.

In the present embodiment, the breathing assistance device 202 is a mechanical ventilator, however, in other embodiments the breathing assistance device 202 may be a PAP or CPAP device. The breathing assistance device 202 includes an inspiratory tube 204 and an expiratory tube 204′ for providing an airflow pathway for airflow leaving and returning to the breathing assistance device 202, respectively. The inspiratory tube 204 and the expiratory tube 204′ may be connected to the breathing assistance device controller 206 at one airflow pathway using the tube connector 214. The airflow may then flow to the user 210 through another airflow pathway of the breathing assistance device controller 206. The airflow from the inspiratory tube 204 may be subjected to perturbation from a forced oscillation produced by a motor or an actuator (hereinafter referred to as an “actuator” to refer to both cases) 216 generating an oscillation of air at a desired frequency and intensity. The actuator 216 may be one of a loud speaker, an electromagnet, a piezoelectric device, a piston and a motor, for example. The choice of actuator may be dependent on the design specifications such as the physical size of the device 206 as well as on the limitations imposed on the Bill of Materials (BOM). It should be noted that in some embodiments, the actuator 216 can be included in the breathing assistance device 202 and not in the breathing assistance device controller 206. Alternatively, in some embodiments, both the breathing assistance device 202 and the breathing assistance device controller 206 can include separate actuators.

In general, the generated oscillation pressure signal may also be controlled to deliver a desired pressure. In some cases, it may be preferable to produce pressures (i.e., amplitude of the generated oscillation signal) that do not exceed a peak-to-peak value of about 0.01 cm H₂O to about 2 cm H₂O. In some other cases, the pressure may be chosen on the basis of the frequency of oscillation or on the sensitivity and precision of the flow rate sensor and/or the pressure sensor.

The inspiratory tube 204 and the expiratory tube 204′ may be combined prior to reaching the user 210 at a junction using a tube fitting 218 connected to a breathing tube 208. Subsequent to the tube fitting 218, the combined airflow may be sensed to determine airflow parameters such as the airflow rate and the air pressure. In this example embodiment, the sensors used comprise a flow transducer 220 and a pressure transducer 221. It should be noted that the flow transducer 220 may also be called a flow rate transducer or an airflow transducer. The sensor type used for the transducers 220 and 221 can be any appropriate transducer device, including but not limited to, ultrasonic, pneumatic or piezoelectric transducers, for example. In some embodiments, the airflow parameters can be measured and calculated by recording the pressure drop across a pneumotachograph, which is used as the sensor.

The outputs of the flow transducer 220 and the pressure transducer 221 may be preconditioned prior to being further processed and analyzed. For example, the output signals from the transducers 220 and 221 may be amplified by an appropriate amplifier 224 to obtain the desired signal amplitudes. For example, in some embodiments, the amplifier 224 may be a lock-in amplifier which may be used to reduce signal noise to help focus on the frequency of interest. It should be noted that separate amplifiers can be used for each measured signal or a dual channel amplifier may be used.

The amplified signal may then be filtered to remove extraneous frequency domain content. The filter may be a low pass filter, a high pass filter, a bandpass filter, or any other appropriate filter.

After the signals have been amplified and filtered, the signals are received by the processor 228 for further processing and analysis in order to apply one or more predictive models in order to generate a control signal 212 that is provided to the breathing assistance device 202 to adjust its operation, as described in more detail below.

In some embodiments, the processor 228 may be a programmable device such as a programmable microcontroller or a field programmable gate array (FPGA). In other embodiments, the processor may be part of a single-board computer system platform such as the Arduino platform, or Raspberry Pi platform. In yet other embodiments, the signal filtering may be performed using the processor 228 such as by using digital signal processing (DSP) techniques such that separate filtering device 226 may not be necessary. In some embodiments, there might be multiple processors that perform dedicated functions.

In the embodiment of FIG. 2, the breathing assistance device controller 206 includes the processor 228. In some embodiments, the breathing assistance device controller 206 can be separate from the processor 228. For example, in some embodiments, the breathing assistance device controller 206 can be physically coupled to the breathing assistance device 202 while in other example embodiments, the breathing assistance device controller 206 can be wirelessly coupled to the breathing assistance device 202, for example, via the network 114. For example, in some embodiments, the breathing assistance device controller 206 can be implemented on a mobile phone, on a separate device located in the same room as the breathing assistance device 202, or on a remotely located device.

The control signal 212 can be provided to the breathing assistance device 202 using any method known to those skilled in the art. For example, the control signal 212 can be provided through a wired connection. However, in other implementations, the control signal may be communicated wirelessly to the breathing assistance device 202 in which case the system 200 can include a transmitter or a transceiver (such as a Wi-Fi or Bluetooth transceiver).

The measured airflow parameters, determined indices and/or control signal 212, may also be shown on an optional display 230 provided on the breathing assistance device controller 206. The display 230 may be, but is not limited to, an LCD display such as that for a tablet device or smartphone. In some embodiments, determined indices may include indices of the user's body position, average temperature and/or one or more of the indices described in U.S. Pat. Nos. 11,633,560 and 11,612,708.

The system 200 also includes a memory unit 229 which can include RAM, ROM, one or more hard drives, one or more flash drives or some other suitable data storage elements such as disk drives, etc. The memory unit 229 stores program instructions for an operating system 229a, a device control module 229b, one or more data files 229c, an input/output (I/O) module 229d, one or more machine learning models 229e, a simulation module 229f and the therapy module 124. The memory 229 may also include device profile data 229g, the user model 120 and the device model 122. The simulation module 229f may be used for performing digital twin simulations, while the device profile data 229g stores data that includes various settings and program instructions for configuring the system 200 to provide operate the breathing assistance device as if it were a CPAP device, an APAP device and the like as described herein. It should be noted that some of these modules may be optional depending on the embodiment. While some of these components are shown residing in a cloud server as in FIG. 1B, in at least one embodiment they may also be stored locally in the system 200 and uploaded to/downloaded from the cloud server when being updated. Other software instructions may be included as is known to those skilled in the art.

The device control module 229b comprises software instructions that, when executed, configures the processor 228 to operate in a particular manner to implement various functions, processes, and methods for the system 200. For example, the device control module 229b can include program instructions for sensing various data from the sensors 223, performing various calculations or processing using the sensed data and then applying one or more of the machine learning models 229e to generate a control signal in order to control the breathing assistance device 202 to provide update the airflow provided to the user, which may be to prevent a respiratory event from occurring and/or providing updated therapy to the user. As another example, the device control module 229b can include program instructions for identifying false negative predictions based on the performance of the machine learning models 229e and extracting false negative data corresponding to the identified false negative predictions, which may be used to update the personalized model for the user.

The machine learning models 229e include different types of models that are used for classifying or predicting different sleep or breathing related phenomena. The machine learning models 229e may be based on using different machine learning methods such as one or more of a Random Forest classifier, a logistic linear classifier, the K-Nearest Neighbors algorithm, the Feature-Based Dissimilarity Space Classifier (FDSC), Neural Networks and support vector machines. In an alternative embodiment, deep learning or Convolutional Neural Networks (CNN) may be used. For reduced computational time, it may be preferable to use a Random Forest classifier as the machine learning model.

One or more of the machine learning models 229c generally also include a trained predictive model that may, for example, be determined on the general population. In at least one embodiment, there may be multiple trained predictive models which may have been determined based on an age group (e.g., deciles such as 31 to 40, 41 to 50, etc.), user sex (e.g., male or female), respiratory condition (e.g., user has severe sleep apnea or moderate sleep apnea, etc.).

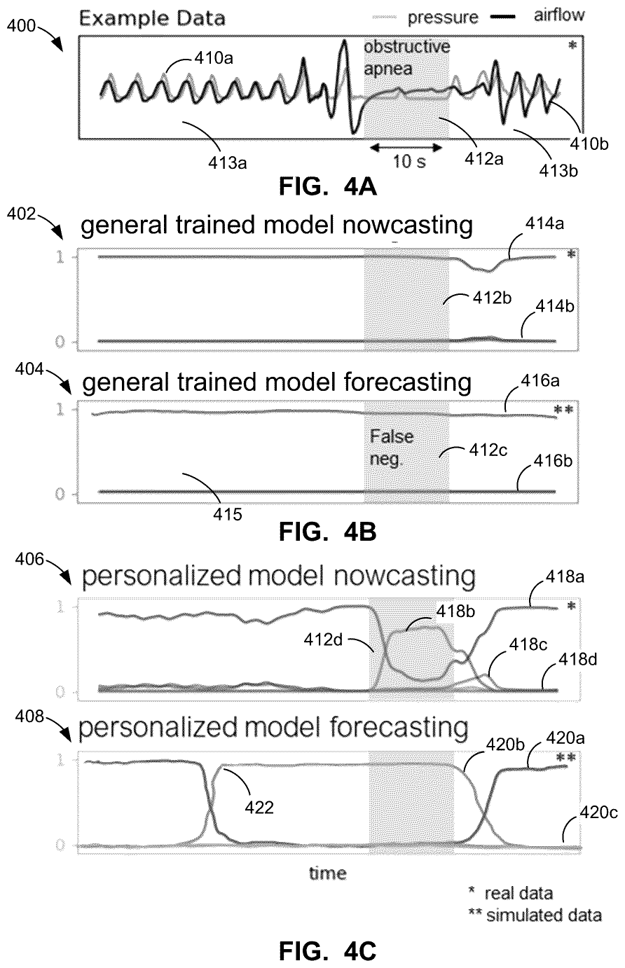

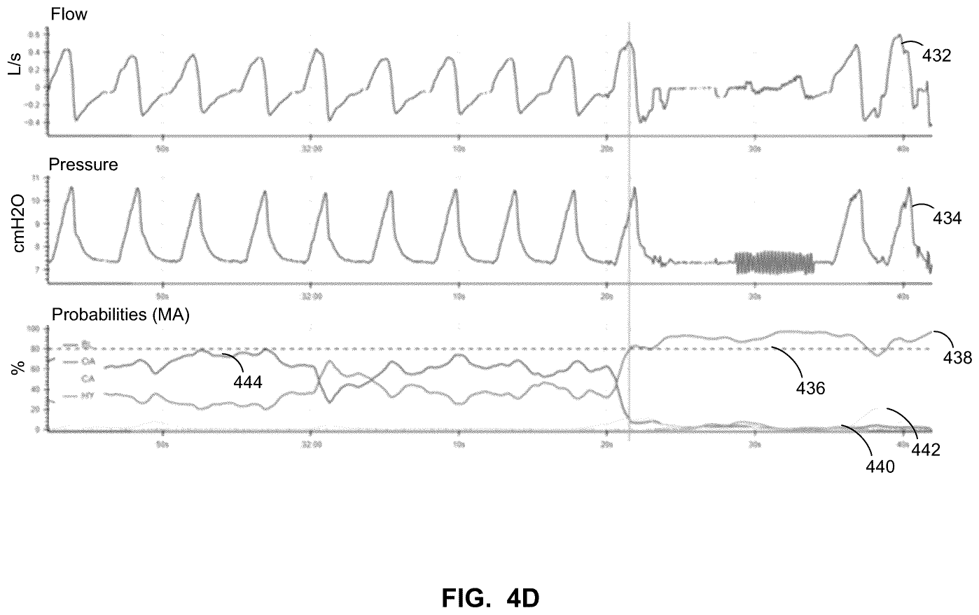

One or more of the machine learning models 229e generally also include a personalized predictive model for adjusting the airflow provided by the breathing assistance device 202 to the user 210, a user model and/or a device model for the breathing assistance device 202. An example embodiment of the personalized predictive model is described in more detail with respect to method 300 in FIG. 3A and the example data in FIGS. 4A-4C. An example embodiment of the models of the user and of the breathing assistance device 202 are described in more detail with reference to FIG. 7.

Certain input features are provided to the machine learning model that have high predictive power. For example, the input features may include the Power Spectral Density (PSD) of the measured air flow (e.g., airflow and airflow rate are synonymous). The PSD may be measured over the entire spectral range of the sampled sensor data. Alternatively, there may be cases where the PSD over a more specific frequency range may be measured such as between about 0.01 Hz to about 20 Hz which contains most of the information for the PSD. Other potential input features may include one or more of the PSD of the measured air pressure, the PSD of the measured resistance and reactance using FOT, and for a given PSD, the number of peaks in the PSD, and the power at a certain frequency. In other cases, the input features for a given measured or determined signal may be one or more of an index for the signal; a windowed linear least squares regression coefficients along the signal; the absolute energy of the signal (e.g. the sum of the squared amplitudes of the signal); the min and/or max amplitude of the signal; the standard deviation, skewness, and/or kurtosis of the signal; the number of peaks in the signal; the autocorrelation of the signal and the absolute value of the FFT coefficients of the signal. The signal may be the measured air flow, the measured air pressure, the reactance obtained from the FOT method, the resistance obtained from the FOT method or the impedance obtained from the FOT method. The actual input features depend on the particular type of machine learning model. For example, in some embodiments, the input provided to the machine learning model includes sensor data measured from one or more sensors described herein.

The machine learning models 229e may be trained in various ways. For example, data obtained from patients may be preprocessed (as described herein for FIG. 2) and divided into a training set, a testing set, and a validation set. For example, the training set, the test set, and the validation set may comprise using amounts of the data in the proportion of 70%, 15% and 15%, respectively. The training data is used to train the machine learning model so that it accurately predicts a desired parameter. The machine learning models may be initialized with some initial parameters and then trained using the training set to determine that machine learning model parameters and input features that provide the highest accuracy. The machine learning model may then be tested with the test data set and the accuracy noted. The parameters of the machine learning model may then be adjusted to maximize the accuracy of the machine learning model on the test data set. The machine learning model may then be tested with the validation data set and the accuracy was noted.

The data used to develop, test, and validate the machine learning models 229e described herein was obtained at the QEII Health Sciences Centre in Halifax. The data included thousands of respiratory failure events comprising sleep apneas of various categories including obstructive, central and hypopnea sleep apnea. The data was split into test, training and validation data sets. In some embodiments, the data used to generate the machine learning models 229e is personalized to the user, for example, the false negative data generated while the user was using the breathing assistance device 202. Obstructive sleep apnea events were extracted from the data, and data was also obtained from baseline and pre-apnea periods. Data was also taken based on different EEG determined sleep stages. The data includes measured pressure of air flow, measured air flow rate, and determined resistance and reactance using the measured pressure and air flow rates while performing the FOT method at different frequencies. The data also included other PSG measurements.

The input/output module 229d receives input data that was obtained by the sensors, preprocesses the input data, and/or provides outputs data (or signals such as control signal 212 from processing done by the device control module 229b) that are then sent to the corresponding hardware. The input/output module 229d may, for example, operate in conjunction with the device control module 229b to communicate data (or signals) between one or more of the processor 228 and one or more of the sensors 220 to 223 and the display 230. In various embodiments, the functionality of the input/output module 229d may be implemented, for example, using a combination of hardware, firmware, and/or software.

The data files 229c may store any temporary data (e.g., data that is not needed after the breathing assistance device 202 has been used) or permanent data (e.g., data saved for later use), such as user data (e.g., a user ID), settings for the breathing assistance device 202, preprocessing or other processing settings including variables and calibration data, and various machine learning models such as at least one personalized model, at least one user model and/or at least one device model. The data files 229c may also include various user data for each user that uses the breathing assistance device 202 such as identification data, respiratory physiological data and recorded user data during use of the breathing assistance device 202. For example, the data files 229c may include files with information for the user model 120, the device model 122 and the device profiles 229g, which are shown separately for clarity.

In at least one embodiment, the breathing assistance device controller 206 can be configured to operate continuously to monitor the pressure and flowrate of the airflow provided to the user 210 to allow for detecting and/or predicting the user's breathing state for constant adjustment of the operation of the breathing assistance device 202. Doing so may permit real-time or near real-time adaptive adjustments to be made to minimize or avoid any respiratory failure that is experienced by the user 210. In other embodiments, the breathing assistance device controller 206 may alternatively be controlled to operate intermittently, for example, at a set time interval. In at least one embodiment, the set time interval may be every few milliseconds, every few seconds, every 30 seconds, or every hour. However, other set time intervals may be used. Such operating conditions may be preferred if the breathing assistance device controller 206 is battery operated so as to help extend the operational lifetime of the breathing assistance device 202.

The sensors may also include at least two additional sensors 222 and 223. Sensor 222 can be a CO2 gas sensor. The sensors 223 can include one or more user sensors that may be placed at certain locations on the user 210. For example, sensors 223 can include sensors that are used to obtain at least one physiological signal and/or at least one neurological signal. For example, the physiological signals include one or more of ECG, EOG, and EMG signals and blood parameters including inflammatory markers (as determined via a blood test which may be provided from data sent from a laboratory or a sensor) and the neurological signals include one or more of EEG and Peripheral Neurophysiological Examination (PNE) signals. These signals can be measured using known electrodes that are placed at certain locations on the user 210 as is known by those skilled in the art. As the position of a user can affect the therapy provided by the breathing assistance device, sensors 223 can additionally include positional sensors placed at certain locations on the user 210 to determine the position of the user. In at least one embodiment, a CO2 signal may be obtained and/or light sensors may also be placed on the user's skin may also be used to measure blood-related parameters. In at least one embodiment, sensors 223 can additionally include environmental sensors, placed at different locations within the room where the breathing assistance device 202 is used. For example, environmental sensors can include: (a) sound sensors such as microphones that can record sounds from the user and/or the environment (e.g., snoring, breathing ambient noise, outside noise); (b) movement detection and/or recording systems including (I) image and video acquisition and/or recording systems, and/or (II) radar and/or electromagnetic recording systems, that can detect and/or record movement (e.g., breathing, sleep position, etc.); (d) humidity sensors; (e) alcohol and/or substance sensors that can measure an alcohol or substance concentration; (f) position sensors, such as gravity sensors, gyroscopes, accelerometers, that can detect a user's position; (g) light sensors that can detect ambient light; (h) pressure sensors placed for example on the surface on which the user is sleeping to measure movement and position; (i) any other sensor that can detect environmental parameters and parameters related to the user; or any operable combination of items (a) to (i). In at least one embodiment, sensors 223 can additionally include device sensors placed on or within the breathing assistance device 202 to measure properties of the breathing assistance device 202 as described previously. These sensors can be used to for example, monitor the health of physical components of the breathing assistance device. For example, a humidity sensor may be placed within the breathing assistance device 202 to monitor a humidity level of the device. The sensor data may be pre-processed as is known by different channels of the amplifier 224 and the filter 226 to reduce noise for these particular signals before these signals are sent to the processor 228 for further analysis. The settings for the amplification and filtering of the signals are known to those skilled in the art.

The system 200 also generally includes a network communication module 232 that enables communication with other devices via a network 114. For example, the breathing assistance device controller 206 can send data to remote devices via the network communication module 232 and the network 114. The data sent to remote devices can include, for example, sensor data, processed data and/or determined indices, false negative data from the one or more machine learning models 229e and models used by the breathing assistance device 202 as described in more detail herein.

Referring now to FIG. 3A, shown therein is a flowchart of an example embodiment of a method 300 for generating a personalized predictive model for adjusting an airflow provided by a breathing assistance device to a user. The method 300 can also be used to receive sensor data and use the sensor data to generate a nowcast and a forecast of the user's breathing based on the personalized predictive model for adjusting the airflow provided by the breathing assistance device 202 to the user. The method 300 can be performed by the processor of the controller 206 when executing software instructions of the various modules described earlier. However, in other embodiments, the method 300 can be performed by other processors or another applicable device. For example, in some embodiments, the method 300 can be performed by a combination of processors including, for example, the processor 228 of the breathing assistance system 200 and a remote processor. For ease of explanation, the elements depicted in the breathing assistance system 200 shall be used in describing the various steps of the method 300. For example, the method 300 may be implemented by the processor 228 of the breathing assistance system 200. However, it should be understood that this technique can be used on the integrated breathing assistance device controller 206 or another applicable device.

The method 300 may begin when the breathing assistance device 202 has been activated, and is supplying an airflow to the user 210. Starting at act 302, a trained predictive model is deployed at the breathing assistance device controller 206. For example, the trained predictive model can be saved to the memory unit 229 of the breathing assistance device controller 206. The trained predictive model can be generated in accordance with the details discussed with reference to FIG. 2. For example, the trained predictive model may be determined on the general population. In at least one embodiment, there may be multiple trained predictive models which may have been determined based on an age group (e.g., deciles such as 31 to 40, 41 to 50, etc.), user sex (e.g., male or female), respiratory condition (e.g., user has severe sleep apnea or moderate sleep apnea, etc.). In at least one embodiment, the trained predictive model can receive the sensor data as input. In at least one embodiment, the trained predictive model can receive determined features as input. The determined features can be determined based on the sensor data. In at least one embodiment, the features are determined based on sensor data from a specific population and used as input to one or more models for a general population. In at least one embodiment, the features are determined based on sensor data of a specific user and used as input to one or more models for that specific user. For example, if it is determined that a threshold of 80% in forecasting of obstructive apnea performs better than 70% in patients after coronary revascularization, or another type of surgery, this feature of having surgery may be in an input to the predictive model. This feature may be tested in a digital twin simulation (further details provided below) on other users that are of the same population and if the results are also good, the update may be applied to all of the population of same age range that have had that surgery. Or if pre-apnea intervention with 3 cm H2O for a period of 3 seconds results in a reduction in AHI for patients with COPD on PAP, this feature may be used as an input to the predictive model and may be applied to other patients that fit the same category. This may be applied to in digital twin simulation. Categorizing patients may happen via clustering, or threshold through signal processing techniques, or machine learning for classification of different populations that the new features apply to. This may be applied in digital twin simulation. Using the sensor data, the trained predictive model can generate a nowcast of the user's current breathing state and a forecast of the user's future breathing state within a predicted time period. In at least one embodiment, the predicted time period is immediately before a breathing event. The predicted time period may be limited by the period of the sampling frequency of data, 1/f seconds, where f is the sampling frequency. In at least one embodiment, the predicted period is the maximum time in which a prediction of an event can reasonably be made. Other predicted time periods may be used. In some embodiments, the trained predictive model receives other types of data as input, such as, for example, processed sensor data or values calculated and/or determined based on the sensor data. This is discussed in more detail with reference to acts 304 and 306 below.

At act 304, sensor data is received from one or more sensors coupled to the breathing assistance device controller 206. In some example embodiments, the sensor data includes flow rate and pressure measured from the flow transducer 220 and the pressure transducer 221, respectively, of the airflow (including the perturbation) that is sent to the user 210. The sensor data may be amplified by the amplifier 224 and filtered by, for example, filter 226. The filter can include a low pass filter, a high pass filter, a bandpass filter, or another appropriate filter. Also noted previously, in some embodiments, the passband may be made sufficiently narrow such that a notch filter can be used instead when a single frequency is used in the FOT measurement. If an FOT measurement is not being performed, the filtering may be applied to generally remove noise as is known by those skilled in the art.

In some embodiments, the sensor data can include data from other sensors such as the gas sensor 222 and/or one or more sensors 223, including PSG sensors. For example, the sensor data can include at least one physiological signal and/or at least one neurological signal as described previously. As another example, the sensor data can include a signal from one or more environmental sensors, as described previously.

After the signals have been processed by applying amplification and filtering, the processed signals are received by the processor 228 for further processing and analysis. For example, in at least one embodiment, the further processing may include processing the signals to have a zero mean and unit variance. In some embodiments, the processor 228 may use the sensor data to calculate and/or determine various values or indices representative of the user's breathing state. For example, in some embodiments, a peak-to-peak value or associated PSD may be calculated. In some embodiments, indices such as indices of the user's body position or average temperature may be calculated. In some embodiments the sensor data is provided as input to the predictive model. In other embodiments, the calculated and/or determined values or indices are provided as input to the predictive model.

At act 306, the trained predictive model is applied to the input data to generate the nowcast and the forecast of the user's breathing state. In some embodiments, the trained predictive model generates a nowcast and a forecast of the user's breathing state for each timepoint of the input data. For example, in at least one embodiment, the trained predictive model is trained on a large set of data to perform classification. The training data, for example, could be split into training, test, and validation data in proportions of 70%, 15%, and 15%, respectively. At each timepoint, the trained predictive model can use a memory of previous timepoints for context to classify the current breathing state and the predicted future breathing state based on the current timepoint of data. The trained predictive model may be developed using various means such as, but not limited to, a convolutional neural network (CNN), a deep neural network (DNN), a long short term memory (LSTM) layers, a Reinforcement Learning (RL) model or an operable combination thereof, for example. Other implementations of the trained predictive model may be used. This generally involves optimizing the architecture of the AI/machine learning model that is selected by determining, e.g., the number of LSTM layers and number of units per layer are determined, and the learning rate parameters (e.g., the initial learning rate, the max learning rate, and the functional form of the learning rate (which drops as training progresses)) that result in acceptable/best performance. In an example embodiment for an LSTM machine learning model, the following parameters were found to result in acceptable performance: LSTM layers: 3 units per layer; 32 max learning rate; 1 initial learning rate; 10e-5; learning rate function (how max learning rate decreases with training time); ½(x−1) where x=training epoch.

In some embodiments, the forecast represents a probability of the user's predicted breathing state within a predicted time period. For example, in some embodiments, the predicted time period can be any one of 20 seconds, 40 seconds, or 60 seconds; however, other predicted time periods may also be used. In some embodiments, the forecast comprises a plurality of probabilities corresponding to a plurality of breathing states such that each breathing state is represented by a probability that the user's future breathing state within the predicted time period will correspond to the respective type of breathing state. Types of future breathing states can include: (a) normal breathing, (b) a respiratory failure event including but not limited to obstructive apnea, central apnea, hypopnea, obstructive hypopnea, and/or respiratory effort related arousal, or (c) unclassified event. Other breathing states and respiratory breathing events may be included. In some embodiments, a normal breathing state comprises a state in which a respiratory failure event is not detected and/or predicted for a certain time period.

In some embodiments, the nowcast represents a probability of the user's current breathing state. In some embodiments, the nowcast comprises a plurality of probabilities corresponding to a plurality of breathing states such that each breathing state is represented by a probability that the user's breathing current state corresponds to the respective type of breathing state. As with the forecast, types of nowcast breathing states can include: (a) normal breathing, (b) a respiratory failure event including obstructive apnea, central apnea, hypopnea, obstructive hypopnea, and/or respiratory effort related arousal, or (c) an unclassified event. Other breathing states and respiratory failure event types may be used. In some embodiments, a normal breathing state comprises a state in which a respiratory failure event is not detected and/or predicted for a certain time period. In some embodiments, the nowcast is generated by determining, based on the input data, a probability that the user's current breathing state (e.g., corresponding to the current time period) comprises a breathing event and a probability that the user's current breathing state comprises normal breathing. In at least one embodiment, the current time period may be the last timepoint, the last 10 timepoints, the timepoints from the last hour, the timepoints from the last day, etc., where a timepoint is a sample. Other current time periods may be used.

In some embodiments, an adjustment in the airflow provided by the breathing assistance device 202 to the user 210 can be made based on the nowcast and/or the forecast. For example, in some embodiments, one or more of the forecasted probabilities and/or nowcasted probabilities can be compared to a threshold. Referring now to FIG. 3B, shown therein is an example flowchart of the flow of data through the breathing assistance system 100. Input data 316 can include, for example, user data 318, medical professional data (e.g., from a physician, healthcare professional, hospital, pharmacy, etc.), breathing assistance device data 320, environment sensor data 322, and/or predetermined thresholds 324. User data 318 can include, for example, any suitable data related to the user 110 of the breathing assistance device 202 as described herein. Some examples of user data include any combination of alcohol consumption, drug consumption, medication, feelings related to being on medication, insomnia, restfulness, tiredness, and the like. Breathing assistance device data 320 can include any suitable data from the breathing assistance device 202 such as, for example, pressure data and/or airflow rate data. Environment sensor data 322 can include, for example, data from one or more environment sensors as described herein such as, but not limited to, humidity of the air in the room, sounds in the room and outside of the room, and/or temperature in the room where the room is where the user is sleeping. Predetermined thresholds 324 can include, for example, thresholds that are preselected based on expected nowcast and/or forecast probabilities (e.g., based on group data of users similar to user 110). Although some types of input data 316 are shown in FIG. 3B, it should be understood that the input data 316 is not so limited and can include other types of input data as described herein and/or any, all, and/or none of the types of input data 316 shown.

The input data can be provided to the predictive model 326 to generate nowcast probabilities 328 and/or forecast probabilities 330 as described herein. The predictive model 326 can include, for example, the trained predictive model and/or the personalized predictive model as described herein (e.g., the personalized predictive model is the trained predictive model after being personalized/further trained to a particular user). The input data 316 can further be provided to one or more threshold models 332, which can determine one or more calculated thresholds 334. For example, the threshold model 332 can apply known signal processing techniques to the input data 316 to determine the one or more calculated thresholds 334. In some embodiments, one or more of the calculated thresholds 334 can be personalized to the user 110. For example, if the threshold of flow limitation, or snoring, or fragmentation, or oxygen drop reaches a certain amount (after filtering the data into the right frequency range after receiving from the sensors) an action may occur which may include reducing or increasing the pressure of the air that is provided to the user.

The nowcast probabilities 328, forecast probabilities 330, calculated thresholds 334, and/or predetermined thresholds 324 can be used for threshold comparisons 336. In some embodiments, one or more threshold comparisons 336 can be performed. For example, FIG. 3B shows three different threshold comparisons 336. In other example embodiments, zero, one, two, or more than three threshold comparisons 336 can be performed. For each threshold comparison 336, one or more of the nowcast probabilities 328 and/or forecast probabilities 330 is compared to the threshold 338. Each of thresholds 338a-338c can be associated with, for example, a different type of respiratory event. The threshold model may be continually operating until the thresholds are personalized to the user. For example, if AHI is below 2 apnea events per hour and the patient's respiratory, cognitive and cardiological health is acceptable, the thresholds may be considered to be personalized (e.g., optimized) for this particular user. Periodically, the thresholds may be checked to determine if changes are needed such as when the user's physiological health has changed such that adjustments are needed for the personalization to be more effective. For example, if health outcomes or one of the sensors that has to do with the health of the heart, brain or respiratory system of the user or even the user's own feedback (e.g., such as through manual entry) shows that they are not doing well, then the thresholds may be adjusted. Based on the comparison, the controller 206 can perform one or more actions. For example, if the comparison to thresholds 338a-c indicate that the nowcast probability 328 and/or the forecast probability 330 meets the threshold condition, the controller 206 can take actions 1A-1C shown at 340a-340c in FIG. 3B, respectively. Alternatively, if the comparison to thresholds 338a-338c indicate that the nowcast probability 328 and/or the forecast probability 330 do not meet the threshold condition, the controller 206 can take actions 2A-2C shown at 342a-324c of FIG. 3B, respectively. Although FIG. 3B shows only one action for each outcome of each threshold comparison, it should be understood that more than one action can be performed. For example, action 1A (340a) can include one or more actions and/or sub-actions. In some embodiments, the one or more actions 340a-340c and 342a-342c can be personalized to the user 110. For example, if a user's/patient's apneas are predicted over 70% probability, then air pressure to the airflow provided to the user increases to prevent the apnea, but if the probability doesn't go over 70% and the probability of now casting shows no change in breathing state, then the air pressure provided to the user remains the same. In another patient that threshold may be 80%. As another example, if nowcasting shows that the probability of snoring is 20% more than usual, then air pressure for the airflow to the user may be increased slightly to open up the user's airway further and prevent snoring. As another example, if probability of central apnea is increasing more than 50%, the pressure of the airflow to the user may be decreased to prevent more central apneas. These thresholds may be different in different users/patients. It should be noted that the terms user and patients may be used interchangeably.

Accordingly, in one or more embodiments, the controller 206 can adjust the airflow provided by the breathing assistance device 202 to the user 210 based on a comparison of the forecast probabilities to a threshold (e.g., a probability threshold). In at least one embodiment, the threshold can be predefined to be a 50%, 70%, or 90% probability, for example. Other predefined thresholds may be used. In some embodiments, the threshold can be a calculated threshold 334 as described herein. For example, in one example embodiment, an adjustment can be triggered when a forecasted probability of at least one respiratory failure event is above a threshold of 75%. As another example, in one example embodiment, an adjustment can be triggered when a forecasted probability of normal breathing is below a threshold of 50%. In at least one embodiment, the threshold is optimized and personalized to the user (for example, various decisions for a specific user may need a specific threshold for that user and the thresholds may be updated/changed if for a particular user if their health state changes). In at least one embodiment, the threshold can be updated at various time periods, for example, in real time (on the order of a few seconds), near-real-time (on the order of a few minutes), hourly, daily, weekly, monthly, etc. Accordingly, the threshold may be adaptive to be more reactive to the user's current sleep behaviour and/or physiological health.

Alternatively, in some embodiments, one or more of the nowcast probabilities can be compared to a threshold. Based on the comparison, the controller 206 can adjust the airflow provided by the breathing assistance device 202 to the user 210. In some embodiments, the threshold can be predefined to be a 50%, 70%, or 90% probability level, for example. Other predefined thresholds may also be used. In some embodiments, the threshold can be a calculated threshold 334 as described herein. For example, in one example embodiment, an adjustment can be triggered when the nowcast probability corresponding to an obstructive apnea breathing event is above a threshold of 75%. As another example, in at least one example embodiment, an adjustment can be triggered when a nowcast probability of normal breathing is below a threshold of 50%. In at least one embodiment, the threshold is optimized and personalized to the user in a similar manner as described above. In at least one embodiment, the threshold can be updated at various time periods, for example, in real time (on the order of a few seconds), near-real-time (on the order of a few minutes), hourly, daily, weekly, monthly, etc. Accordingly, the threshold may be adaptive to be more reactive to the user's current sleep behaviour and/or physiological health.

In at least one embodiment, the various thresholds that are used may be different for each breathing state, e.g., the normal breathing state and each of the respiratory breathing stages, and these thresholds may also be personalized to each user. These various thresholds may also change over time. Adjusting the various thresholds in this manner by the controller or remote process allows for more personalized therapy to be provided to the user.

In at least one embodiment, both the nowcast and the forecast probabilities can be used to determine a control signal for the controller 206. For example, in at least one embodiment, the nowcast and the forecast can have respective weights representative of the importance of the respective value to the control signal. For example, in at least one embodiment, the nowcast may be more critical to determining the airflow to be provided to the user by the breathing assistance device 202 and accordingly, the nowcast will have a higher weight than the forecast when determining the control signal for the controller 206. As another example, in at least one embodiment, the forecast may be more critical to determining the airflow to be provided to the user by the breathing assistance device 202 and accordingly, the forecast will have a higher weight than the nowcast when determining the control signal for the controller 206. The combination of the weighted nowcast and forecast may then be compared to a threshold for making decisions.

Returning now to FIG. 3A, at act 308, one or more false negative predictions may be identified. A false negative prediction occurs when the nowcast and/or the forecast fails to correctly indicate a current or future respiratory failure event when the user indeed experienced a breathing event. In some embodiments, identifying that the user experienced a respiratory failure event can be determined, for example, based on the sensor data. For example, a false negative prediction of the nowcast may be identified when the sensor data indicates that the user experienced a respiratory event at a current time period and the nowcast indicated a normal breathing state of the user at the current time period. In another example embodiment, a false negative prediction of the forecast may be identified where the sensor data indicates that the user experienced a respiratory failure event at a current time period and the forecast indicated a normal breathing state within a predicted time period, where the predicted time period corresponds to the current time period. A given breathing state can be indicated based on, for example, the comparison to a threshold, in accordance with the description above.

In other example embodiments, a false negative prediction occurs when one or more of the nowcast or forecast fails to correctly indicate a particular respiratory failure event type. For example, a false negative prediction of the nowcast may be identified when the sensor data indicates that the user experienced an obstructive apnea breathing event at a current time period and the nowcast indicated a central apnea breathing event of the user at the current time period. In another example, a false negative prediction of the forecast may be identified where the sensor data indicates that the user experienced an obstructive apnea breathing event at a current time period and the forecast indicated a central apnea breathing event within a predicted time period, where the predicted time period corresponds to the current time period. A given breathing state can be indicated based on, for example, the comparison to a threshold, in accordance with the description above.

At act 310, false negative data is received. In some embodiments, the false negative data is received at a processor or device that is remote from the breathing assistance device controller 206. For example, in some embodiments, the breathing assistance device controller 206 sends the false negative data to a server 118 via the network communication module 232 and the network 114. The false negative data comprises, for each of the one or more false negative predictions, the nowcast, the forecast, and a portion of the sensor data used for determining the nowcast and forecast. In some embodiments, the portion of sensor data extends from a first time point before an onset of the false negative prediction to a second time point after the offset of the false negative prediction. In some embodiments, the first time point and the second time point are in the range of 20 seconds to 60 seconds. However, other first time points and second time points may be used.

In some embodiments, the onset of a false negative prediction is defined as the first time point of a predetermined number of consecutive false negative nowcasts and/or forecasts. For example, in some embodiments, the onset of a false negative prediction is the first time point of the first false negative nowcast and/or forecast of three consecutive nowcasts and/or forecasts. Other predetermined numbers of consecutive false negative nowcasts and/or forecasts may be used.

In other embodiments, the onset of a false negative prediction is determined to be the time point at which the controller 206 generates a control signal 212, for example, to adjust or maintain the airflow provided to the breathing assistance device 202 based on the generated nowcast and/or forecast. In some embodiments, the offset of a false negative prediction is defined as the time point at which a predetermined number of consecutive correct nowcasts and/or forecasts are generated. For example, in some embodiments, the predetermined number of consecutive correct nowcasts and/or forecasts can be three. Other predetermined numbers of consecutive correct nowcasts and/or forecasts may be used.

In some embodiments, the false negative data is pre-processed. In at least one embodiment, the false negative data is pre-processed to generate a cleaner set of data for use in training or re-training predictive models. Pre-processing the false negative data can include one or more of: normalization using techniques known to the skilled person, averaging the portion of sensor data of the false negative data, principal component analysis, independent component analysis, down sampling, up sampling, frequency filtering, or manual inspection of the portion of sensor data of the false negative data.

At act 312, the personalized predictive model is generated by re-training the trained predictive model using the false negative data. The personalized predictive model is personalized to the user. Features identified in sensor data can differ from user to user. Accordingly, a predictive model that is trained using group data only may not account for certain sensor data features that are unique to a given user. A personalized predictive model that is re-trained using data from a specific user can improve detection and/or prediction performance of the model for that user.

In some embodiments, the personalized predictive model is generated/retrained automatically at a predetermined frequency. For example, in some embodiments, the predetermined frequency can be in real time, ever few minutes, hourly, daily, weekly, or monthly. Other predetermined frequencies may be used for how often the personalized predictive model is retrained.

In some embodiments, the personalized predictive model is generated after a minimum number of false negative predictions are identified. In an example embodiment, the minimum number of false negative predictions can be 500 and the personalized predictive model is generated once at least 500 false negative predictions are identified. In some embodiments, the minimum number of false negative predictions can be in the range of one to the total number of time points at which a nowcast and/or forecast is generated during a monitoring time period in which the breathing assistance device is used by the user. Other minimum number of false negative predictions may be used.

In some embodiments, the personalized predictive model is generated after a predetermined period of time has elapsed. For example, in some embodiments, the personalized predictive model is generated daily, weekly, or monthly. Other predetermined periods of time may be used.

The performance of the predictive model generally improves with the amount of data used for training. In some embodiments, it is preferable to have larger amounts of false negative data to re-train the trained predictive model to generate the personalized predictive model. In some embodiments, the method 300 comprises generating simulated false negative data for re-training the trained predictive model. For example, in some embodiments, simulated false negative data may be generated when the number of identified false negative predictions is less than the minimum number of false negative predictions. The simulated false negative data can be generated by applying one or more signal processing techniques to the sensor data. In some embodiments, the one or more signal processing techniques are selected to preserve at least some of the information of the sensor data. For example, the one or more signal processing techniques can include applying jittering, noise addition, magnitude scaling, or chunk truncating. The skilled person in the art will appreciate that other techniques of simulating data are possible.

However, in at least one embodiment, a larger amount of false negative data is not needed to re-train the trained predictive model. In some embodiments, a portion of the false negative data for a specific user may no longer be relevant (e.g., due to a particular classification of the user, e.g., phenotype) and may be omitted during re-training. In at least one embodiment, such omission of data occurs when the user's amount of false negative data is sufficiently large.

In some embodiments, re-training the trained predictive model includes one or more of performing transfer learning, tuning one or more parameters of the trained predictive model, adding one or more layers to the trained predictive model, performing reinforcement learning or any combination thereof. In some embodiments, re-training the predictive model further uses at least some of the sensor data. For example, in some embodiments, re-training the predictive model uses the sensor data (e.g., data from any of the pressure sensors, flow sensors, PSG sensors, environmental sensors, etc.) in addition to the false negative data.

Transfer learning can include training the predictive model from scratch using the false negative data. In example embodiments using transfer learning, the model weights are re-learned, and the model architecture is preserved.

Tuning one or more parameters of the trained predictive model can include continuation or resumption of the training used to initially train the trained predictive model, but using only the false negative data. In example embodiments using this technique, the model weights of the trained predictive model are not altered significantly, and the model architecture is preserved. The trained predictive model may include one or more nodes and one or more layers, and the one or more parameters that may be tuned include a type of node, a selection of nodes, a node weight, a node activation, a node memory, a number of connections between nodes, an orientation of connections between nodes, an orientation of connections between layers, a type of layer, a number of layers, a connection between layers, a number of inputs, a number of outputs, or an operable combination thereof, for example. Other parameters known to a skilled person may be used.

Adding one or more layers to the trained predictive model can include, for example, adding an output layer to the model thereby altering the architecture of the model. In at least one embodiment, adding one or more layers to the trained predictive model can include adding one or more intermediate layers. In example embodiments using this technique, the model weights of the trained predictive model are not altered, and the weights associated with the additional output layer are learned by way of the training using the false negative data.

Reinforcement learning can include training the predictive model to maximize a cumulative reward. For example, the predictive model can have one or more states represented by a time series window of physiological data, such as airflow data of the user. At each timestep, a decision made by the predictive model (e.g., predicted value of the user's airflow data) can change the state of the predictive model. The predictive model can receive a reward at each timestep for the decision made by the predictive model. Accordingly, at each timestep, the predictive model may use the airflow data (e.g., airflow pressure data) of the user as input to ultimately output a probability distribution of all possible airflow data values (e.g., all possible airflow pressure values) for the user. The airflow data value (e.g., airflow pressure value) with the highest probability is selected by the predictive model as the next timestep airflow data value (e.g., airflow pressure value). The predictive model then transitions to the next state (e.g., timestep) and receives a reward for the completed timestep. The RL may be performed for training to maximize the cumulative reward received over the course of an episode with T timesteps. The training may be done to obtain personalized models that maintain normal breathing, improve health metrics and/or decrease air pressure needed for sleep therapy.

In some embodiments, reinforcement learning can include implementing a Q learning technique. In such cases, for pressure management, at any given time Q values (airflow, set pressure) are estimated to optimize metrics of interest. For example, for any moment, given an airflow window, there may be 100 (4.0-14.0, step size 0.1) possible pressure values to choose from which means for the current airflow state there are 100 Q values to estimate. Any airflow state in the dataset for pressure management, will have 100 possible Q values to estimate. As the model is trained it will get better at estimating the Q values (a value to represent the expected future reward or a value to represent the likelihood that a certain pressure value is the optimal pressure).

In some embodiments, reinforcement learning can include implementing a deep-Q learning technique. Traditionally Q values are calculated and kept track of in a big (state, action) table. However, as the number of possible states becomes incredibly large, this is not feasible. For pressure management, the number of possible states/airflow windows may become incredibly large. To address this issue, deep-Q learning may be used which involves using neural networks as Q value estimators. For pressure management, a neural network takes the airflow window as input and outputs the probability distribution over all possible pressure values and estimates the optimal pressure to maximize future reward/metrics of interest.

In some embodiments, reinforcement learning can include implementing DDQN learning, which may be an improvement on the DQN approach. The DDQN approach uses two neural networks where one of them is updated randomly to tackle overestimation issues with the original DQN.

In some embodiments, reinforcement learning is performed offline (e.g., training and/or re-training the predictive model is based on previously collected data through offline reinforcement learning). Traditional/Online RL methods are based on an online learning paradigm, in which an algorithm/agent actively interacts with an environment. However, performing offline RL may be more efficient and also ethical, since patients at not put risk by training and making adjustments to the personal predictive model in real-time. Since there may be few optimal transitions in offline datasets, and many unseen transitions, CQL (Conservative Offline RL) training may be used to minimize overestimation of Q values on unseen transitions.

In some embodiments, the offline TL training may use a training dataset (state, action rewards) based on past airflow windows and pressure changes, from which rewards are determined for all (state, action) pairs. For apneas and flow limitations, negative rewards may be determined based on the reward function to optimize metrics of interest. For example, CQL with a deep learning model may be for training to predict the pressure values that maximizes future rewards. This training may also use different techniques to address bias towards a policy used in existing data and improve generalization to unseen data.

The skilled person in the art will appreciate that other re-training techniques are possible.

In some embodiments, the personalized predictive model is generated by conditioning the trained predictive model using a summary representation for the user. The summary representation comprises user data. In some embodiments, the summary representation is generated based on data generated the day before. In some embodiments, the summary representation is an average representation based on an exponential moving average of the summary representation. This allows for in-context adaptation of the trained predictive model using existing parameters, without requiring re-training or tuning of the parameters. Conditioning the trained predictive model using the summary representation of the user can include changing how the parameters used for inference and training may influence the classification and/or learning. The summary representation may be used to alter the behaviour of the trained predictive model without re-training the trained predictive model.

In some embodiments, the summary representation includes user characteristics such as weight, height, gender, sex, age, body mass index, apnea-hypopnea index, SpO2, mask type of the breathing assistance device, prescribed pressure to be provided by the breathing assistance device, location type, location elevation or any operable combination thereof.

In some embodiments, the summary representation includes one or more statistical representations of the user's breathing based on the sensor data. The one or more statistical representations of the user's breathing can include an average waveform of a breath of the user, a variance for each sample timepoint within the average waveform, or one or more of a minimum, maximum, average, median, or variance of the user's air flow, air pressure, tidal volume, respiratory rate, SpO2, heart rate, sound and/or motion, or any operable combination thereof.

In some embodiments, the summary representation includes one or more statistical representations of the user's environment based on the sensor data. The one or more statistical representations of the user's environment can include one or more of a minimum, maximum, average, median, or variance of one or more of temperature, ambient CO2, or ambient O2.

In some embodiments, the summary representation includes statistical representations of the user characteristics, user breathing, and/or user environment.

In some embodiments, the summary representation can be generated by the trained predictive model. An embedding layer of the model can be generated by a block within the model with learnable parameters based on the summary representation, where the block is optimized for the task of creating an embedding layer. The embedding layer can learn and change how the summary representation impacts the trained predictive model. A block within the model can include, for example, a single layer or a plurality of layers.

In some embodiments, the trained predictive model is conditioned on the summary representation by providing the summary representation as input to the trained predictive model in addition to the sensor data. In other embodiments, the trained predictive model is conditioned on the summary representation by adapting a feature representation based on cross-attention with the summary representation. The feature representation is based on the sensor data. In other embodiments, the trained predictive model is conditioned on the summary representation by providing the summary representation as input to a normalization block characterized by an offset factor and a scale factor for each feature corresponding to the normalization block. The offset factor and the scale factor can be determined by a machine learning model conditioned on the summary representation. In at least one embodiment, the features are abstracted, and may not be known prior to conditioning. In some embodiments, the machine learning model is a multilayer perceptron.

At act 314, the personalized predictive model is deployed to the breathing assistance device controller 206. In embodiments where the false negative data is received at a processor or device that is remote from the breathing assistance device controller 206 and the personalized predictive model is generated/updated at the device, the personalized predictive model can then be sent/deployed to the breathing assistance device controller 206 via the network communication module 232 and the network 114. For example, the personalized predictive model may be generated at the server 118, sent via the network 114 to the network communication module 232 and deployed on the controller 206.

In some embodiments, the personalized predictive model is updated at the breathing assistance device controller 206 and deployed thereat automatically at a predetermined update frequency. For example, in some embodiments, the predetermined update frequency can be in real time, near-real time, every few minutes, hourly, daily, weekly, or monthly. Other predetermined frequencies may also be used.

In some embodiments, the personalized predictive model determines one or more of a pressure increase rate, a pressure decrease rate, and/or a pressure amplitude for adjusting the airflow provided by the breathing assistance device to the user. In such embodiments, the pressure increase rate, pressure decrease rate, and/or pressure amplitude can be personalized to the user. For example, some users may require a faster pressure increase rate and/or a higher pressure amplitude than other users (e.g., some users may need a faster/slower ramp-up or ramp-down rate for the change in airflow and/or higher/lower airflow amplitudes.

In some embodiments, the method 300 may include an additional step of monitoring the performance of using the personalized predictive model to determine whether it is not performing in a safe operating rate and if it is not performing in a safe operating range, avoiding the use of the personalized predictive model which may include operating based on OEM settings while the personalized predictive model is retuned so that it's use results in safe operation of the breathing assistance device. This may be done by recording one or more operational characteristics and comparing the recorded operational characteristic(s) to a safety threshold or safety range. For example, if the number of respiratory failure events are recorded, compared to a respiratory failure event safety threshold and found to be above the threshold, it can be determined that the personalized predictive model results in the breathing assistance device 202 to be outside of a safe operating range and use of the personalized predictive model can be disabled. In alternative embodiments, two or more parameters may be monitored and compared to safety thresholds such that when each of the two or more parameters are outside of safe operating ranges. In at least on embodiment, the sensitivity, precision, F1 score, and/or adjusted F1 score of the personalized predictive model, which are described in more details below, may be used for these safety comparisons. In at least one embodiment, if the simulations of the digital the twin (described below) show that the associated sensitivities of upcoming apneas for the user are dropping to under 50%, while the sensitivity for the user used to be over 65%, it can be determined that the personalized predictive model results in the breathing assistance device to be outside of a safe operating range and the therapy provided by the breathing assistance device 202 can be downgraded to a safe version until personalization for the user is safely bringing the sensitivity back to 65%, as determined by digital twin simulations, at which point an over the air update can be pushed.

Referring now to FIG. 4A shown therein is example sensor data 400 that is processed in accordance with at least one embodiment disclosed herein where a general trained prediction model is used. In this example, the sensor data comprises pressure sensor data 410a and airflow sensor data 410b. In other examples, embodiments may be used in which the sensor data can comprise data measured by other types of sensors, as discussed above. It can be seen from the example shown in FIG. 4A that the user's breathing state includes an obstructive apnea breathing event during time period 412a. It can further be seen from the example embodiment shown in FIG. 4A that the user's breathing state includes a normal breathing state during time periods 413a and 413b.

Referring now to FIG. 4B, shown therein is an example nowcast 402 and an example forecast 404 generated by a general trained model using the sensor data 200 shown in FIG. 4A. In the embodiment used for this example, the nowcast comprises a probability 414a, for each timepoint of the sensor data, that the user's breathing state comprises normal breathing at each given timepoint. In the embodiment used for this example, the nowcast further comprises a probability 414b, for each timepoint of the sensor data, that the user's breathing state comprises a respiratory failure event at each given timepoint. In at least one embodiment, the probabilities of more than one breathing event types is near zero and accordingly, may overlap on a visualization of the probabilities. The probability of a breathing type that exceeds the threshold is the breathing type that is identified for that breathing event.

In this example, the forecast comprises a probability 416a, for each timepoint of the sensor data, that the user's future breathing state within a predicted time period will comprise normal breathing. In this example embodiment, the forecast further comprises a probability 416b, for each timepoint of the sensor data, that the user's future breathing state within a predicted time period will comprise a respiratory failure event.

In the example shown in FIG. 4B, it can be seen that the nowcast 402 fails to correctly detect the obstructive apnea breathing event identified in the sensor data shown in FIG. 4A. Time period 412b of the nowcast 402 indicates that the user's breathing state comprises normal breathing at the time of the obstructive apnea breathing event 412a identified in the sensor data 400. In the example shown in FIG. 4B, it can further be seen that the forecast 404 fails to correctly predict the obstructive apnea breathing event identified in the sensor data shown in FIG. 4A. Time period 415 of the forecast 404, occurring a predicted time period before the obstructive apnea breathing event 412a identified in the sensor data, indicates that the user's breathing state will comprise normal breathing at the time of the obstructive apnea breathing event 412c.

Referring now to FIG. 4C, shown therein is an example nowcast 406 and an example forecast 408 generated by a personalized predictive model using the sensor data shown in FIG. 4A. In this example, the nowcast comprises a probability 418a, for each timepoint of the sensor data, that the user's breathing state comprises normal breathing at each given timepoint. In this example, the nowcast further comprises a probability 418b, for each timepoint of the sensor data, that the user's breathing state comprises an obstructive apnea breathing event at each given timepoint. In this example, the nowcast further comprises probabilities 418c-d, for each timepoint of the sensor data, that the user's breathing state comprises other types of breathing type events at each given time point.

In this example, the forecast comprises a probability 420a, for each timepoint of the sensor data, that the user's future breathing state within a predicted time period will comprise normal breathing. In this example, the forecast further comprises a probability 420b, for each timepoint of the sensor data, that the user's future breathing state within a predicted time period will comprise an obstructive apnea breathing event. In this example, the forecast further comprises probabilities 420c, for each time point of the sensor data, that the user's future breathing state within a predicted time period will comprise other types of breathing event types at each given time point.

In the example shown in FIG. 4C, it can be seen that the nowcast correctly detects the obstructive apnea breathing event identified in the sensor data shown in FIG. 4A. Time period 412d of the nowcast indicates that the user's breathing state comprises an obstructive apnea breathing event corresponding to the obstructive apnea breathing event identified in the sensor data in FIG. 4A.

In the example embodiment shown in FIG. 4C, it can further be seen that at timepoint 422, the forecast correctly predicts the obstructive apnea breathing event that will occur within the predicted time period corresponding to the obstructive apnea breathing event identified in the sensor data in FIG. 4A.

Accordingly, it can be seen from FIGS. 4A to 4C that the personalized predictive model provides improved detection and prediction of the user's breathing state relative to the general trained predictive model generated from non-personalized data (i.e., from general population data).

Referring now to FIG. 4D, shown therein is example flow data 432 and pressure data 434. Also shown therein is example probability data determined from an example forecast generated by an example personalized predictive model. This example probability data includes probabilities for each timepoint of the flow and pressure data, that the user's future breathing state within a predicted time period will comprise an obstructive apnea breathing event 438, a central apnea event 442, a hypopnea event 440, or normal breathing 444. This example probability data may be compared to a threshold 436 to make a decision.

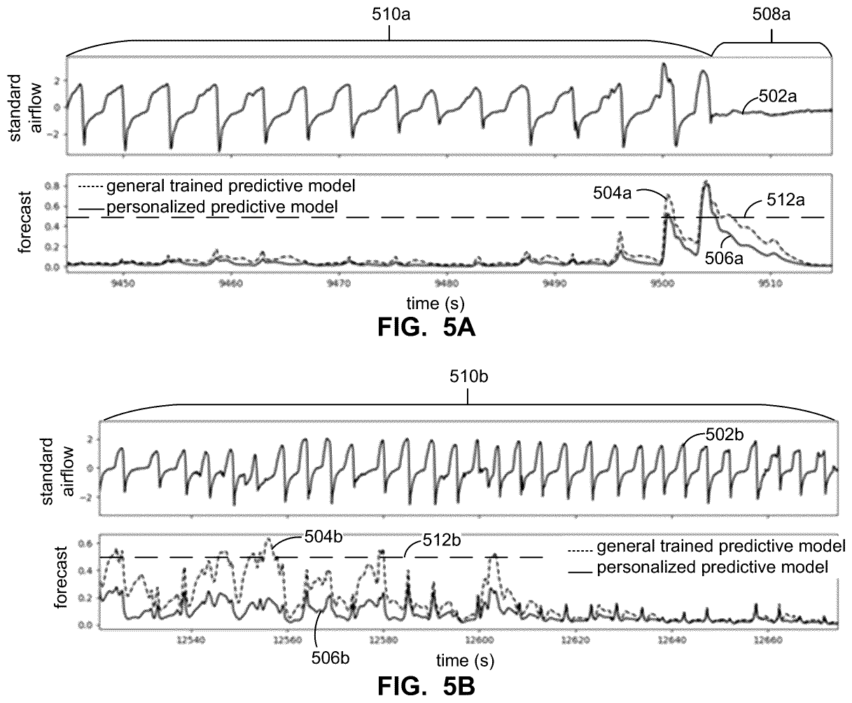

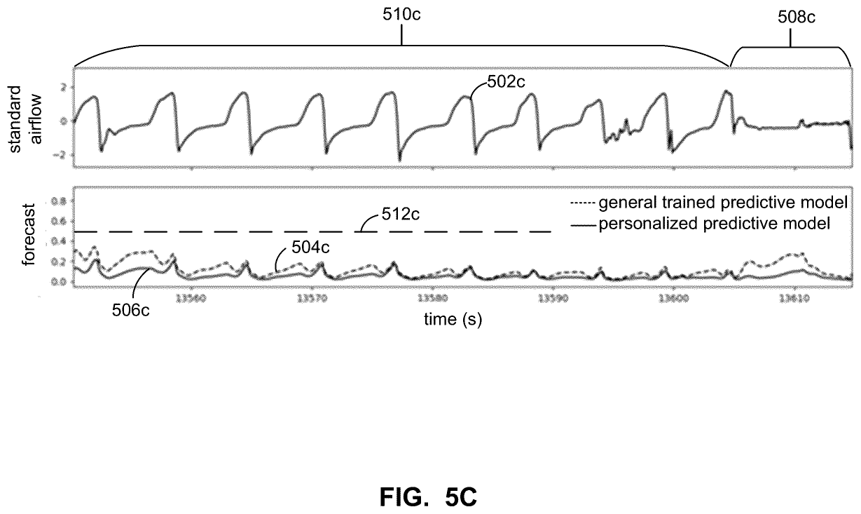

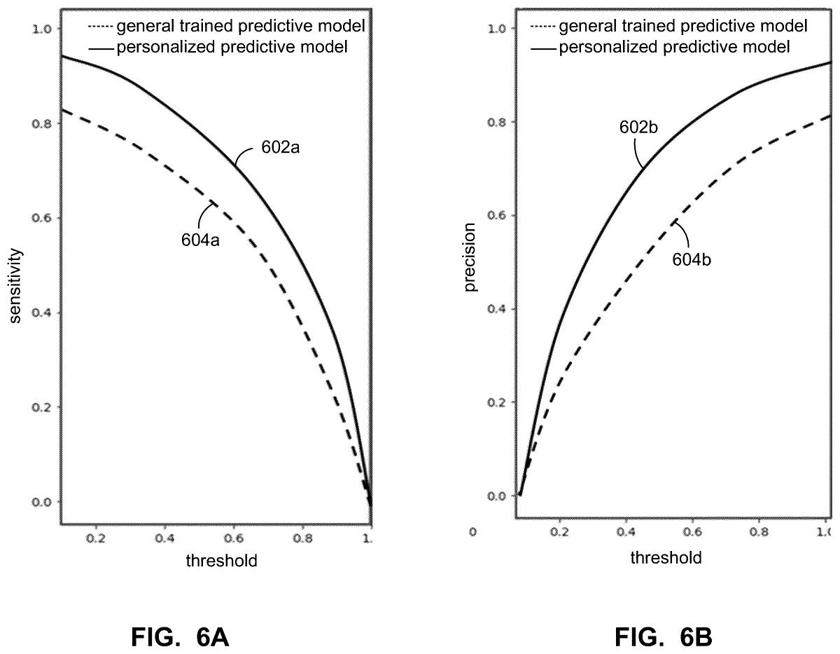

Reference will now be made to FIGS. 5A, 5B, 5C, 6A, and 6B. In some embodiments, a sensitivity, a precision, an F1 score, and/or an adjusted F1 score can be determined for the general trained predictive model and/or the personalized predictive model. Sensitivity may also be referred to as recall or true positive rate and determined by True Positives divided by the sum or true positives and false negatives). For example, the sensitivity can be determined by dividing the number of correct predictions of a respiratory failure event by a predictive model (e.g., true positives) by the total number of actual respiratory failure events (e.g., sum of true positives and false negatives). Accordingly, reducing the number of false negatives of a predictive model increases the sensitivity of the predictive model. The precision can be determined by dividing the number of correct predictions of a respiratory failure event by a predictive model (e.g., true positives) by the total number of respiratory failure events predicted by the predictive model (e.g., sum of true positives and false positives). Accordingly, reducing the number of false positives of a predictive model increases the precision of the predictive model. The F1 score can be determined based on the harmonic mean of sensitivity and precision of a predictive model. For example, the F1 score can be determined based on the following equation:

$$F 1 = 2 \times \frac{precision \times sensitivity}{precision + sensitivity} \qquad ( 1 )$$

Accordingly, a predictive model with a low precision and/or a low sensitivity will also have a low F1 score. The adjusted F1 score can be determined by applying one or more adjustments to the F1 score calculation as described, based on a bias and/or imbalance in the data. For example, if in a specific data set if precision matters more than sensitivity, a weight may be applied to precision. An unbalanced data set may result from having data in too many of one class and not enough in the other class so weights may be applied in this case.

Accordingly, the sensitivity, precision, F1 score, and/or adjusted F1 score can provide an indication of the performance of a predictive model. In some embodiments, the personalized predictive model can be updated based on one or more of the sensitivity, precision, F1 score, and/or adjusted F1 score of the general trained predictive model and/or the personalized predictive model. For example, in some embodiments, the personalized predictive model can be re-trained and/or one or more parameters of the personalized predictive model can be updated based on one or more of the sensitivity, precision, F1 score, and/or adjusted F1 score. In some embodiments, the personalized predictive model can be generated based on one or more of the sensitivity, the precision, the F1 score and/or the adjusted F1 score of the general trained predictive model. For example, if a threshold changes from 40% to 60% for a user, and a higher adjusted F1 score is achieved, then that higher threshold may be used for that user as it will likely result in better outcomes.

Referring now to FIG. 5A, shown therein is example sensor data comprising airflow sensor data 502a. In other examples, embodiments may be used in which the sensor data can comprise data measured by other types of sensors, as discussed above. It can be seen from the example shown in FIG. 5A that the user's breathing state includes an apnea breathing event during time period 508a. It can further be seen from the example embodiment shown in FIG. 5A that the user's breathing state includes a normal breathing state during time period 510a.

FIG. 5A further shows example forecast data 504a generated by an example general trained predictive model using the sensor data 502a and example forecast data 506a generated by an example personalized predictive model using the sensor data 502a. In the embodiment used for this example, the forecast data 504a comprises a probability for each timepoint of the sensor data 502a that the user's future breathing state within a predicted time period will comprise a respiratory failure event based on the example general trained predictive model. In the embodiment used for this example, the forecast data 506a comprises a probability for each timepoint of the sensor data 502a that the user's future breathing state within a predicted time period will comprise a respiratory failure event based on the example personalized predictive model.

In this example, a threshold 512a of 0.5 is shown. In other example embodiments, other threshold levels can be used. A probability represented by the forecast data 504a or 506a that reaches or exceeds the threshold 512a indicates that the respective predictive model predicts that the user's future breathing state within the predicted time period will comprise a respiratory failure event. As shown in FIG. 5A, the forecast data 504a and 506a each exceeds the threshold 512a before the respiratory failure event at time period 508a. Accordingly, each of the general trained predictive model and the personalized predictive model correctly predicted the respiratory failure event that occurs at time period 508a (e.g., true positive prediction).

Referring now to FIG. 5B, shown therein is example sensor data comprising airflow sensor data 502b. In other examples, embodiments may be used in which the sensor data can comprise data measured by other types of sensors, as discussed above. It can be seen from the example shown in FIG. 5B that the user's breathing state includes a normal breathing state for the duration of the time period 510b shown.

FIG. 5B further shows example forecast data 504b generated by an example general trained predictive model using the sensor data 502b and example forecast data 506b generated by an example personalized predictive model using the sensor data 502b. In the embodiment used for this example, the forecast data 504b comprises a probability for each timepoint of the sensor data 502b that the user's future breathing state within a predicted time period will comprise a respiratory failure event based on the example general trained predictive model. In the embodiment used for this example, the forecast data 506b comprises a probability for each timepoint of the sensor data 502b that the user's future breathing state within a predicted time period will comprise a respiratory failure event based on the example personalized predictive model.

In this example, a threshold 512b of 0.5 is shown. In other example embodiments, other threshold levels can be used. A probability represented by the forecast data 504b or 506b that reaches or exceeds the threshold 512b indicates that the respective predictive model predicts that the user's future breathing state within the predicted time period will comprise a respiratory failure event. As shown in FIG. 5B, the forecast data 504b exceeds the threshold 512b at several instances throughout time period 510b indicating a prediction that the user's future breathing state within the predicted time period will comprise a respiratory failure event. However, as shown by the sensor data 502b, the user's breathing state in this example does not comprise a respiratory failure event. Accordingly, the general trained predictive model incorrectly predicted one or more respiratory failure events (e.g. false positive prediction). As shown in FIG. 5B, the forecast data 506b does not reach or exceed the threshold 512b throughout time period 510b indicating a prediction that the user's future breathing state within the predicted time period will comprise normal breathing. Accordingly, the personalized predictive model correctly predicted normal breathing (e.g., true negative prediction).

Referring now to FIG. 5C, shown therein is example sensor data comprising airflow sensor data 502c. In other examples, embodiments may be used in which the sensor data can comprise data measured by other types of sensors, as discussed above. It can be seen from the example shown in FIG. 5C that the user's breathing state includes an apnea breathing event during time period 508c. It can further be seen from the example embodiment shown in FIG. 5C that the user's breathing state includes a normal breathing state during time period 510c.

FIG. 5C further shows example forecast data 504c generated by an example general trained predictive model using the sensor data 502c and example forecast data 506c generated by an example personalized predictive model using the sensor data 502c. In the embodiment used for this example, the forecast data 504c comprises a probability for each timepoint of the sensor data 502c that the user's future breathing state within a predicted time period will comprise a respiratory failure event based on the example general trained predictive model. In the embodiment used for this example, the forecast data 506c comprises a probability for each timepoint of the sensor data 502c that the user's future breathing state within a predicted time period will comprise a respiratory failure event based on the example personalized predictive model.

In this example, a threshold 512c of 0.5 is shown. In other example embodiments, other threshold levels can be used. A probability represented by the forecast data 504c or 506c that reaches or exceeds the threshold 512c indicates that the respective predictive model predicts that the user's future breathing state within the predicted time period will comprise a respiratory failure event. As shown in FIG. 5C, neither forecast data 504c nor 506a reaches the threshold 512c before the respiratory failure event at time period 508c. Accordingly, each of the general trained predictive model and the personalized predictive model failed to correctly predict the respiratory failure event that occurs at time period 508c (e.g., false negative prediction).

Referring now to FIG. 6A, shown therein is example sensitivity data 602a corresponding to an example personalized predictive model and example sensitivity data 604a corresponding to an example general trained predictive model. The sensitivity data 602a and 604a represent the number of correctly predicted respiratory failure events (e.g., true positives) by the personalized predictive model and the general trained predictive model, respectively, divided by the total number of respiratory failure events that occurred within a measured time period. FIG. 6A shows the sensitivity data 602a and 604a as a function of threshold, where the threshold represents the probability at or above which the corresponding forecasting model predicts that the user's future breathing state within the predicted time period will comprise a respiratory failure event. As shown in FIG. 6A, the personalized predictive model has a better sensitivity than the general trained predictive model at all thresholds.

Referring now to FIG. 6B, shown therein is example precision data 602b corresponding to an example personalized predictive model and example precision data 604b corresponding to an example general trained predictive model where these models are similar to those of FIG. 6A. This is just one example of an optimal threshold that may be chosen to find the best precision and sensitivity on the same patient. The precision data 602b and 604b represent the number of correctly predicted respiratory failure events (e.g., true positives) by the personalized predictive model and the general trained predictive model, respectively, divided by the total number of respiratory failure events predicted by the personalized predictive model and the general trained predictive model, respectively (e.g., sum of true positives and false positives). FIG. 6B shows the precision data 602b and 604b as a function of threshold, where the threshold represents the probability at or above which the corresponding forecasting model predicts that the user's future breathing state within the predicted time period will comprise a respiratory failure event. As shown in FIG. 6B, the personalized predictive model has a better precision than the general trained predictive model at all thresholds.

Referring now to FIG. 7, shown therein is a flowchart of an example embodiment of a method 700 for simulating an operation of the breathing assistance device and/or a health state of a user receiving assistance from the breathing assistance device such as, for example, breathing assistance device 102. The method 700 may be used to implement a digital twin simulation of one or more aspects of the user and/or breathing assistance device operation. The method 700 may be used in combination with the method 300 or as an alternative to the method 300. Similar to method 300, the method 700 can be performed by the processor of the controller 206 when executing software instructions of the various modules described earlier. However, in other embodiments, the method 700 can be performed by other processors or another applicable device. For example, in some embodiments, the method 700 can be performed by a combination of processors including, for example, the processor 228 of the breathing assistance system 200 and a remote processor, such as the processor(s) of server 118. For ease of explanation, the elements depicted in FIGS. 1A-2 shall be used in describing the various steps of the method 700. For example, the method 700 may be implemented by the processor 228 of breathing assistance device controller 206. However, it should be understood that this technique can be used on another applicable device.

The method 700 may begin when the breathing assistance device 202 has been activated though in some cases, some steps of method 700 may be performed prior to the breathing assistance device 202 being activated. For example, as will be described, at least some information about the user can be obtained prior to the breathing assistance 202 being activated.

At act 702, a model of the user is received. The user model 120 of the user may be stored, for example, in a data storage such as data storage 116 and/or generated by the server 118 and may be a personalized model or a generic model (if the functionalities provided by method 700 are used for the first time), for example. As described above, the user model 120 is a digital user model which can be termed a “digital twin” of the user and may be a model that can model a current state of the user and a future state of the user. In some cases, the parameters of the model(s) may be initially determined and/or updated from models associated with other individuals. For example, parameters of user models and/or user models associated with multiple other individuals may be collected and stored, for example in data storage 116 and may be distributed to initialize the user model 120. The user models stored can be categorized according to similarities between the individuals with which these user models 120 are associated. For example, the user models can be grouped according to user “phenotypes”. The parameters of the user model(s) can also be used to update parameters of user models associated with other individuals that are similarity grouped. The user model 120 may be a single personalized model or a combination of user models that model different internal systems of the user (i.e., user physiological system models), including but not limited to, the respiratory system, the cardiovascular system, the nervous system of the user or an operable combination thereof, for example and that model the environment of the user as it affects the state of health of the user (i.e., environmental models).

The user model(s) 120 may be implementing using any type of model including mathematical models and neural networks, other machine learning models, or a combination of models. For example, the respiratory system may be modeled as the airway opening in series with a single compartment, two compartments or a multi-branch respiratory tree using an RLC circuit, a mass spring damper system, a non-linear complex network, etc. Similarly, the nervous system model may be an electrical and/or mechanical model, a mathematical model, a neural network, etc., that models sleep stages, a respiratory rate, a ventilatory rate, a heart function and/or other brain-related functions of a user. The cardiovascular system model may be an electrical and/or mechanical model, a mathematical model and/or a neural network that can model cardiovascular functions of a user. In some cases, the models may include models known in the art, for example, models as described in “An integrative model of respiratory and cardiovascular control in sleep-disordered breathing”, Limei Cheng, Olga Ivanova, Hsing-Hua Fan, Michael C. K. Khoo, doi:10.1016/j.resp.2010.06.001 and in “Analysis of Mathematical Models of the Human Lung”, Racheal L. Cooper, Master of Science, Virginia Commonwealth University.

In some embodiments, the user model 120 can be associated with a corresponding physical model (termed a “physical twin”) modeling one or more physiological systems of the user. For example, the user model 120 can be associated with a bench top lung simulator and/or a bench top lung-brain-cardiovascular simulator, which can simulate the user's lung, brain and/or cardiovascular function. Examples of such physical simulators are provided in U.S. Pat. No. 11,610,513 entitled “Benchtop within-breath dynamic lung simulator”, which is incorporated herein in its entirety. The user model 120 and the physical model associated with the user model can be used in combination to simulate a current and/or expected health state of the user or a user's expected response to a therapy/change in therapy. For example, the physical model can be used to validate and/or supplement the user model 120.

In at least one embodiment, the user system models that are used in the user model may be common to all user models, but the parameters of the user system models may be modifiable such that the user model 120 can be personalized to a given user and model the state of health of given user. For example, when sensor data is received, as will be described in further detail below, parameters of the models may be modified such that an expected health state of the user may be determined via simulation. The parameters may be updated by transmitting the sensor data as inputs into the user model(s) 120 and/or the device model(s) 122 to train or re-train the models. The parameters may be updated frequently, as sensor data is received. Alternatively, the parameters may be updated at a slower regular interval such as, for example, hourly, nightly, weekly or by request of the user or a health professional supervising the user's treatment. For example, sensor data may be recorded, optionally preprocessed, and stored as it is received, and the model(s) may be trained to determine the updated parameters each time an update is scheduled and/or requested. The magnitude of change in the parameters may vary depending on the type(s) of model(s) used, the amount of data (e.g., sensor data) received by the model(s), the learning rate of the model(s), the weight of the training classes used by the model(s) and/or other characteristics of the model(s) used.

In at least one embodiment, the model parameters may additionally be personalized based on personal characteristics of the user. For example, when configuring the breathing assistance device 202 or using the functionalities of method 700 for the first time or in some cases, immediately prior to using the breathing assistance device 202, the user may be prompted to provide or update personal characteristics (also called personal user factors), for example, user alcohol consumption, user drug consumption, medication taken by the user, recent blood test results, user height, user weight and/or user age. As these factors may affect a state of health of the user, personalizing the user model 120 using one or more of these personal user factors can allow for more personalization of the user model 120 so that the user model 120 may be a more accurate reflection of the physiological user systems. The user may provide the personal user factors via, for example, the computing device 150 (user computing device 150a in FIG. 1B). In some embodiments, one or more personal user factors can be provided by an external user or an external system, such as, a medical professional overseeing the user's therapy, a pharmacy system, a payor system, a health system, other home medical equipment or an electronic medical records system, can provide one or more personal user factors about the user, via external user computing device 150b (shown in FIG. 1B). In at least one embodiment, prior to or immediately following use of the breathing assistance device 202, the user may be prompted to enter information about the user's current state for example, the user's alcohol or substance consumption, a subjective assessment of the pressure exerted by the breathing assistance device 202 on the user's face (e.g., nasal passages and/or mouth), a subjective assessment of the humidity and temperature of the air provided by the breathing assistance device 202, an assessment of the quality of sleep of the user, or any operable combination thereof. Information provided by the user may be used to vary the weight of the parameters and/or vary the range of parameters that can be used for the overall user model 120 and/or one or more of the user's physiological system models.

In some cases, a generic model based on one or more of the personal characteristics of the user may be used. For example, data storage 116 may include models associated with different categories such as, but not limited to, age categories, sex categories, or weight categories, for example. The models may be derived for example, based on existing models associated with users having similar personal characteristics (e.g., physiological conditions, age, sex, etc.). For example, simulation results from users may be transmitted to a server, such as server 118, which analyzes the results to derive parameters for the model of the user.

At act 704, a device model 122 of the breathing assistance device 202 is received. Similar to the user model 120, the device model 122 of the breathing assistance device 202 may be stored, for example, in a data storage such as data storage 116 and/or generated by server 118 and may be a model that is customized to the breathing assistance device 202 being used or a generic model based on a general type of the breathing assistance device, for example, if the functionalities provided by method 700 are used for the first time. As described above, the device model can be termed a “digital twin” of the breathing assistance device 202. The device model 122 of the breathing assistance device 202 can be a model or a combination of models that model the operation of the breathing assistance device 202. For example, the device model 122 of the breathing assistance device 202 may be based on a combination of device sub-models such as: (a) individual device components of the breathing assistance device 202 such as a piston, a blower, an electrical input of the device, (b) device subsystems of the breathing assistance device 202 such as a humidifier subsystem of the breathing assistance device 202, (c) device functionalities of the breathing assistance device 202 such as the air pressure of the air delivered, the humidity of the air delivered and the airflow of the air delivered, or (d) any operable combination of (a) to (c). Each of these models may be any type of model that can model the operation, components and/or subsystems of a breathing assistance device 202 including, but not limited to an electrical and/or mechanical models, complex transfer functions, neural networks, other machine-learning models, etc.

Similar to the user model 120, the device models 122 used may be common to all breathing assistance device models 122, but the parameters of the device 122 models may be modifiable such that the device model 122 can be customized to the device being used. For example, when sensor data is received, as will be described in further detail below, parameters of the device models 122 may be modified such that an expected performance or operation of the breathing assistance device 202 may be determined. It is possible that for two devices that are the same type (i.e., same manufacturer and model), the devices may behave differently over time due to amount of usage, amount of maintenance, amount of component variation and the like.

At act 706, sensor data is received from sensor(s), for example sensors 220-223. As described with reference to FIG. 2, sensor data may be received from one or more sensors placed at different locations on the user, sensors placed at various locations in the room where the breathing assistance device 202 is located and/or sensors located on or within the breathing assistance device 202. In some embodiments, sensor data can be received from all of the sensors 220-23 available. In other embodiments, sensor data can be received from only some of the sensors available. For example, sensor data from some of the sensors 220-223 may only be received if selected by the user (or an external user). As another example, the processor can select sensors from which to receive sensor based on personal characteristics of the user, a previous operation of the breathing assistance device 202 and/or user feedback. For example, prior to activating the breathing assistance device 202, the user can provide feedback about a previous experience with the breathing assistance device 202. Based on the user feedback received, one or more sensors 220-223 can be selected as sensors from which to receive sensor data. Sensor data may be received as the sensor data is collected, for example, sensor data may be continuously received in real-time. Alternatively, sensor data can be received at predetermined intervals of time.

At act 708, an expected state of the breathing assistance device 202 and/or an expected health state of the user is determined based on the device model 122 of the breathing assistance device 202 and/or the user model 120, as well as the sensor data received at act 706. The expected state may correspond to a future expected state or a current expected state based at least on sensor data received at a previous time. By determining an expected state of the health state of the user and/or of the breathing assistance device 202, changes in a health state of the user or unexpected changes in the operation of the breathing assistance device 202 may be identified. Remedial actions may then be performed if the expected states indicate that an issue is occurring or will occur such as imminent device failure, in which case maintenance may be performed on the device including replacing one or more device components, replacing one or more device subsystems, recalibrating the device or any operable combination thereof.

By using the sensor data received at act 706, the parameters of the user model 120 and/or the parameters of the device model 122 of the breathing assistance device 202 can be modified, such that one or both of the models can be personalized/customized to the user and/or the particular breathing assistance device 202 that is actually used. For example, using sensor data, the parameters of the user model 120 may be modified to adjust the user model 120 to reflect the user characteristics of the user (e.g., the current state of those characteristics). Similarly, using sensor data, the parameters of the device model 122 may be modified, to reflect a current state of the breathing assistance device 202. For example, based on sensor data received, it may be determined that one or more components of the breathing assistance device 202 are not operating according to their original manufacturer specifications. In this case, the parameters of the device model 122 may be modified such that the device model 122 more accurately reflects/models the current state of the breathing assistance device 202. The model(s) may then be used to simulate a current and/or future state of the breathing assistance device and/or of the health of the user over time.

By modeling a current state of the breathing assistance device 202, a future state of the breathing assistance device 202 may be determined. For example, the average lifespan of a component of the breathing assistance device 202 may be known, and based on the current state of the component, including for example, the current age of the component and a current use of the component, the operation of the component over time may be simulated using the overall device model 122 or a component model. For example, based on sensor data received and/or the personal user factors received, the model(s) (e.g., device model 122, component model, user model 120, a combination of the models) may generate synthetic data, which may be used to further train the model(s) (i.e., update model parameters). The synthetic data may be generated by extrapolating current data over time and/or by applying a model such as a regression mathematical model to the current data. Alternatively, synthetic data may not be generated, and a predictive model may be applied directly to the model parameters to update the model parameters based on predictions relating to the model parameters and/or the models themselves. In some cases, the expected state of the breathing assistance device 202 may be determined based in part on the user model 120. For example, it may be determined that certain characteristics of the user affect the operation of the breathing assistance device 202. An asthmatic user for example, may be more sensitive to air quality. In such cases, the lifespan of the air filter of the breathing assistance device 202 may be determined to be shorter than for a non-asthmatic user. Alternatively, or in addition thereto, the environment in which user uses the breathing assistance device 202 may affect the expected state of the breathing assistance device 202. For example, based on sensor data from environmental sensors, it may be determined that the room where the breathing assistance device 202 is placed is at a high elevation or has a poor air quality (e.g., due to smoke, pets, etc.), which may affect the lifespan of some components of the breathing assistance device 202.

The health state of the user may be similarly determined using the user model 120 and optionally, the device model 122 of the breathing assistance device 202. For example, based on the sensor data received at act 704 and the user model 120, the health state of the user may be determined by providing the sensor data as input to the user model 120, since some measurements obtained from sensors may indicate poor health or conversely, good health. For example, some FOT measurements, sound measurements and reactance measurements may be associated with individuals having CPOD. Alternatively, or in addition thereto, the user model 120 may be used to simulate the health state of the user over a certain period of time such as hourly (e.g., for users with very poor health on a ventilator), weeks, months, or another time frame. As described above, based on sensor data received and/or the personal user factors received, synthetic data, which may be used to further train the model(s) and/or predicted model parameters may also be generated and/or a predicted behavior of the model may also be determined. Using the trained models, the current and predicted future health state of the user may then be determined.

In at least some embodiments, the health state of the user can include a sleep health of the user. The sleep health of a user may correspond to a measure of user's sleep health and may be determined based on sleep parameters, including but not limited to, the duration of sleep, the length of the stages of sleep, the depth of sleep at each of the stages and/or the heart rate of the user during sleep, and may be provided as inputs to the user model 120 which can then output a sleep health for the user. The measure may be a score, for example, a unified score based on two or more parameters measured, or a multivariate score (i.e., a score for each parameter). Alternatively, the sleep health may correspond to a set of values corresponding to the parameters measured. Using the sensor data from air pressure sensors and EEG, for example, as parameters to the model(s), one or more of these factors may be evaluated to determine a sleep health of the user. Based on the simulated assessment of the sleep health of the user, the operation of the breathing assistance device 202 may be modified if the simulated sleep health is poor, as will be described in further detail below. The sleep health of the user may also be determined by simulating the health state of the user over time using the models. For example, it may be determined that based on current sensor data, the user is unlikely to achieve deep sleep.

The models of the user and/or of the breathing assistance device 202 may be used to make predictions about the future health state of the user and/or of the breathing assistance device 202. For example, using the user model 120 and optionally the device model 122, the health of the user, which may include the sleep health of the user may be simulated over time and health indicators may be identified. For example, health indicators may correspond to data (e.g., sensor data, synthetic data), patterns of data and/or changes that are known to be associated with a respiratory disease. By simulating the health of the user over time, and identifying health indicators, the occurrence of a respiratory disease may be predicted. For example, based on the identified health indicators, a relative score (e.g., a probability) may be assigned to health conditions or diseases associated with the health indicators. These health indicators may correspond to known indicators and/or to indicators determined from assessing a health state of other users over time. For example, variation over time of a model parameter, discrepancies between an actual and an expected parameter, and/or deviations relative to an expected parameter for an individual having the personal characteristics of the user (e.g., age, sex, weight, respiratory resistance, etc.) may be an indication that the user is developing COPD or a heart disease. An expected parameter value may be determined based on the personal characteristics of the user and/or a previous state of health of the user and deviations from the expected parameter value may be indicative of a health condition. When it is determined that the user is experiencing a respiratory failure event or other health event sensor data received during the event may be transmitted to an external database, such that indicators of health events may be determined over time. Accordingly, in at least one embodiment, various diseases may be predicted or determined based on sensor data such that each disease may be given a relative score for probability. Since data is obtained when the users are using their breathing assistance devices, parameters corresponding to these diseases may be being fitted to the data, and these parameters may then be compared to those from the general population to determine a probability of at least one of these diseases. In embodiments where the user model 120 is associated with a physical model, at least part of the simulation can be done on the physical model.

As another example, the performance of the breathing assistance device 202 may be simulated over time, using the user model 120 and optionally the device model 122. Predicting the future health state of the user can involve in some cases, predicting a sleep disruption. For example, by using user model 120 and optionally the device model 122 to simulate the sleep health of the user over time, it may be determined that a sleep disruption is expected to occur. For example, the depth of sleep of the user and/or a percentage of REM sleep of the user, a determinant of the quality of sleep of an individual, may be measured. By measuring these parameters, the expected percentage of REM sleep of the user may be determined and compared to the expected percentage of REM sleep for individuals having similar or the same personal characteristics as the user, and a deviation between the actual percentage of REM sleep of the user and the expected percentage of REM sleep may be determined. To prevent the sleep disruption or decrease the likelihood of the sleep disruption occurring, the operation of the breathing assistance device 202 may be modified, as will be described in further detail below.

A recommendation may be generated based on the results of the simulation. For example, a recommendation may be made to pre-emptively replace a component of the breathing assistance device 202 and/or perform maintenance, if it is predicted that the component will break down of a device subsystem will fail based on the simulation. Alternatively, or in addition thereto, a report may be generated. The recommendation can be determined by the therapy module 124 which can analyze the results of the user model 120 and the device model 122. Various examples of analysis that may be performed to provide recommendations are provided herein and may be performed by the therapy module 124 by making comparisons between certain data and thresholds or providing certain data to models. In another example, the therapy module 124 may make a recommendation such as sending a message to a physician/health system to prescribe a medication or to ask for permission to make a change to the sleep therapy being provided to the user by the breathing assistance device, as explained further below

In at least one embodiment, the user model 120 and optionally the device model 122 may be used to determine if a change, for example, an unexpected change, has occurred in the health state of the user and/or the state of the breathing assistance device 202. For example, the method 700 can involve simulating the operation of the breathing assistance device 202 over a time period to determine (via simulation) expected parameters associated with the operation of the breathing assistance device 202. The method 700 can then involve receiving sensor data from the sensor(s) and comparing the expected parameters from simulation with actual parameters that are based on the sensor data received. Based on the comparison, a failure of a component of the breathing assistance device 202 or a malfunction of the device 202 may be determined. For example, when the breathing assistance device 202 is simulated as not performing as expected, this can indicate that one or more components of the device 202 will experience a fault. As another example, a difference between the expected airflow delivered, as determined by the control signal 112 and the actual airflow of gasses delivered may indicate that a component is malfunctioning. In such cases, a notification may be generated to alert the user of the fault. In some cases, the alert may indicate the source of the fault and/or identify the component(s) that are malfunctioning.

In some cases, a change in a health state of the user can be indicative of a respiratory disease, a heart disease, a brain disease or any other disease that can be identified using sensor(s) and one or more user models 120. To identify a change in a health state of the user, the method 700 can involve simulating a health state of the user using the user model 120 and optionally the device model 122 of the breathing assistance device 202 over time to determine expected parameters associated with the health state of the user. The method can then involve receiving sensor data from the sensor(s) and comparing the expected parameters with parameters based on the sensor data received. Alternatively, based on the expected parameters determined by the simulations, expected (e.g., simulated) sensor data may be obtained, and the expected sensor data may be compared with the actual sensor data received. For example, a test such as FOT may be performed which indicates that the patient is developing asthma or COPD. In another example, the heart rate of the patient may be analyzed in the digital twin simulation for heart rate variation, and it may be determined that the patient's cardiovascular health is being influenced by the sleep therapy being provided by the breathing assistance device and a change in the sleep therapy may be needed.

Based on the comparison, a recommendation may be generated. For example, if the sensor data received indicates that the health state of the user is lower than the expected health state, for example, as determined by the expected parameter values, an alert notifying the user's health may be generated. Alternatively, or in addition thereto, a health recommendation for improving the health of the user, a diagnosis of a respiratory or other physiological condition, a report indicating a health of the user and/or a health recommendation to consult a medical professional may be generated. For example, if prior to using the breathing assistance device 102, the user indicates consumption of alcohol, and it is determined that the sleep health of the user is affected, the recommendation can include a recommendation about the use of alcohol. In cases where the breathing assistance device 202 is used as part of a treatment supervised by a medical professional, reports may be generated and transmitted to a computing device that is used by the medical professional, for example computing device 150. The recommendation may additionally include a recommendation for using a type of mask, a recommendation for changing the fitting of an existing mask, a recommendation for a new tube for the breathing assistance device 202, recommended settings for the breathing assistance device 202, recommended environmental settings (e.g., room temperature, light exposure, mattress, etc.), or any operable combination thereof. For example, a message may be sent through to a user via the user computing device 150 that their mask has aged and as a result air leakage during use has increased which reduces effectiveness of sleep therapy. The message may also include information that the user's insurance covers the cost of a new mask and that the user should get a new mask. This message may be sent based on analysis performed to determine that the mask needs to be replaced. For example, based on the digital twin simulations and analysis of sound data and air pressure leakage from the mask, a recommendation may be made that the user should change their mask to another specific type of mask that may result in less air leak and better sleep therapy.

For example, during the simulation of the digital twin it may be determined that the breathing assistance device is functioning properly, but due to a bad mask fit, there is a high level of air leakage In this example, recommendation can be provided to the user and/or a medical facility (e.g., shop) where they obtained their mask that the user should change their mask or adjust their mask so that there is a better fit and a reduced air leak. This may be important since in the first weeks of use of a new breathing assistance device, users typically find that they have to go back to the facility where they received the new device and keep asking for adjustments until they feel comfortable using the breathing assistance device, which can be cumbersome and cause some users to give up using the device. However, with the use of the simulation providing automated personalized recommendations, users will be able to more easily adjust the operation of their breathing assistance device and/or use more appropriate equipment (e.g., better fitting masks) which will increase the likelihood that the user will continue to use their breathing assistance device.

In at least one embodiment, the recommendation may involve adjusting the operation of the breathing assistance device 202. For example, as described previously with reference to FIGS. 1A-1B, operational parameters of the breathing assistance device 202 may be adjusted to adjust, correct and/or improve a therapy provided to the user. Since the user model 120 is personalized to the user, the adjustment is customized to the user. For example, based on the user model 120 of the user, it may be determined that the air pressure of the breathing assistance device 202 is causing the user to experience cardiac inflammation. Accordingly, the air pressure of the breathing assistance device 202 may be adjusted (e.g., reduced). As another example, it may be determined that the air pressure of the breathing assistance device 202 is too low. However, it may be determined, based on the user model 120 that the user recently underwent heart surgery (e.g., based on medical records received from a medical professional treating the user or an electronic medical records system or based on medication taken by the user) and that consequently, moderate air pressure instead of high air pressure is to be used.

It should be noted that in general, in any embodiment described herein both real and simulated, adjustments may be made to other factors besides airflow rate and airflow pressure where examples of these factors include, but are not limited to, humidification level, temperature of heated air, dispensing of medication doses in the airflow, for oxygen therapy the oxygen pressure, flow and frequency of application. In the example of medication doses, a medical practitioner may send a message to the user to take a certain dose of medication, which may be included in the airflow by the device such as including statin when Ang2 levels for the user are considered to be high.

As another example, adjusting, correcting and/or improving a therapy provided to the user can include changing a mode of operation of the breathing assistance device 202 by using a device profile that will be more effective in improving the sleep health of the user. For example, a breathing assistance device, with the same hardware, may be configured to operate using a difference device profile (e.g., implemented by software programs and parameter values) so that the breathing assistance device, with no change in hardware, can be configured to operate like several different breathing assistance devices based on the selected device profile. For example, the device profiles include but are not limited to a CPAP, APAP, Bi-PAP, ASV, and non-invasive ventilator NIV device profiles. The breathing assistance device 202 may be operating according to a given device profile and then the breathing assistance device may be re-configured through software to operate according to a different device profile. For example, the device profile of the breathing assistance device 202 may be switched/changed so that it no longer operates as a CPAP device but now operates as a BiPAP device. Accordingly, in at least one embodiment, the control signal 112 may be automatically generated according to the device profile that is selected. In some cases, to adjust the operation of the breathing assistance device 202, a correction factor may be determined and the operation of the breathing assistance device 202, with the correction factor applied, may be simulated using the device model 122 of the breathing assistance device 202 and optionally the user model 120.

As described previously, the models (e.g., device models 122, user models 120) may be configured such that during simulation an expected value of a parameter based on current sensor data, synthetic data and/or predicted model parameters is determined. In at least one embodiment, by simulating the operation of the breathing assistance device 202, expected parameter values may be obtained and a simulated effect of changing the correction factor to the control signal, or using a particular device profile or making some other change, such as updating the personalized prediction model, on the breathing assistance device and/or on the user may be assessed. If it is determined that application of the correction factor and/or other changes achieve the desired effect, then the change to the correction factor may be applied via the control signal 112 and/or the other changes may be made. Conversely, if it is determined that the correction factor does not achieve the desired effect, the correction factor may be modified and the effect of the modified correction factor and/or other changes may be simulated again, using the model(s), until a satisfactory correct factor is found. For example, various correction factors applied to levels of inspiratory and expiratory air pressures, humidity, and/or gasses may be simulated using the models of the breathing assistance device 202 and the user and the impact of these correction factors may be determined based on the simulations.

In at least one embodiment, as sensor data may be continuously received, the adjustment of the operation of the breathing assistance device 202, may take into account the recently received sensor data. For example, based on positional sensor data, it may be determined that the user is on their back and accordingly, the model(s) may simulate the effect of the therapy provided by the breathing assistance device when a user is on their back. In some embodiments, as shown in FIG. 1B, prior to adjusting the operation of the breathing assistance device 202, the breathing assistance device 202 can transmit a request to an external device, for example, the external user computing device 150b associated with a medical professional supervising the therapy of the user and require an indication of approval of the adjustment. In some embodiments where the user model 120 is associated with a physical model, the adjustment can be simulated on the physical model to determine the effect of the adjustment prior to the operation of the breathing assistance device 202 being adjusted.

Once the operation of the breathing assistance device 202 is adjusted, the user's response to the change in operation of the breathing assistance device 202 can be monitored. The user's response to the change in operation can be monitored by evaluating an Intervention Index (II) that characterizes the user's probability of experiencing an upcoming respiratory event in a given hour and/or a Respiratory Event Index (REI), which characterizes the number of respiratory events experienced by the user in a given hour. If the intervention index exceeds a predetermined threshold and/or the respiratory event index exceeds a mean historic number of respiratory events per hour experienced by the user by a predetermined threshold, the breathing assistance controller 206 can revert the adjustment. Alternatively, the breathing assistance controller 206 can change the operation of the breathing assistance device 202 so that the breathing assistance device 202 provides reactive therapy rather than pre-emptive therapy. The intervention index can be determined based on the user model 120 and the mean historic number of respiratory events can be retrieved from memory, for example, from the memory unit 229 or the data storage 116. The activities described in this paragraph can be performed by the digital twin simulation and changes may also be made to the personal prediction model by sending an over the air update to any user's personal predictive model to improve the safety.

In some embodiments, the change in operation of the breathing assistance device 202 and the user's response to the change in operation can be used to train user models associated with other individuals so that similar changes can be applied to individuals associated with similar user models as the user model 120 with the expectation that these similar changes for the other users will achieve similar results. For example, as explained previously, individuals can be classified according to “phenotypes”. A change applied to the operation of the breathing assistance device 202 of one user that achieves favorable results (e.g., improved sleep health) can be implemented on breathing assistance devices associated with other individuals associated with the same phenotype and/or can be used to train the user models associated with other individuals associated with the same phenotype. The activities described in this paragraph can be performed by the digital twin simulation and changes may also be made to the personal prediction model by sending an over the air update to any user's personal predictive model to improve the safety.

For example, patient phenotypes may be developed based on similarities in a combination of data received, age height and outcomes. For example, if a patient with a certain height, age, medication intake, inflammatory biomarker level, and BMI is being treated and their outcomes are similar to another patient with similar height, weight, etc., then those patients may cluster together in terms of these phenotypes and the digital twin simulation may allow for model and operational parameters, that may be saved in a database (e.g., a digital twin database), to apply the same updates to the patients/user fitting that phenotype. Therefore, personalization may happen with groups of patients in some cases. However, further personalization may also be performed according to the teachings herein.

Accordingly, in at least one embodiment, for a given user, the digital twin simulation can be configured to search through the digital twin database to identify an equivalent patient and replicate their breathing solution for this user based on similarities between the given user and equivalent patient.

In another aspect, in accordance with the teachings herein, user (e.g., patient) identification may be incorporated in the digital twin simulation for security/authentication purposes. For example, the user's breathing signature may be determined. This may be done, for example, according to the techniques described in U.S. Pat. No. 11,612,708. The digital twin simulation may then use the user's breathing signature as a security measure to confirm that the patient data and personalized models that are used for digital twin simulation for a given user actually correspond to the given user. For example, the breathing signature may be included with the user's data and personalized predictive model that are provided for digital twin simulation. Separately, part of the digital twin simulation may involve accessing a database to obtain a stored breathing signature for the given user. The stored breathing signature from the database is compared to the received breathing signature that was included with the user data and personalized predictive model to make sure that there is a match before simulation continues. Likewise, the user's breathing signature may be saved with any results from the simulation for security purposes to make sure that future use of data is with the user who has a matching breathing signature.

In another aspect, in accordance with the teachings herein, digital twin simulation may be performed for a given user using a personalized predictive model to simulate how well the personalized predictive model will perform when it is actually deployed for use by user's breathing assistance device. Accordingly, a personalized predictive model may first be determined for the user using any of the techniques described herein. Digital twin simulation is then performed by incorporating the personalized predictive model into the device models that are used to simulate the operation of the breathing assistance device. Physiological data for the user from the simulation data may then be analyzed to determine the effect on health of the user when using the personalized predictive model and optionally simulating the operation of the breathing assistance device according to a selected device profile as explained previously. If the health effect is determined to be beneficial, which may involve determining that the user is not expected to develop a health condition or has a certain quality of sleep compared to normative data, then the personalized prediction model is determined to be beneficial and is then deployed for use with the user's actual breathing assistance device. In at least one embodiment, false negative data that is determined for the user, which may be done according to the teachings herein, is also included in the digital twin simulation so that the simulation may be more realistic.

In at least one embodiment, the digital twin simulation may simulate the ongoing use of the personalized predictive model, the selected device profile and/or any other operational parameters to determine when the breathing assistance is no longer effective (e.g., the user's sleep health declines) in which case use of another personalized predictive model, the selected device profile and/or any other operational parameters may be simulated to determine changes to one or more of these items that will achieve improved sleep health for the user.

In at least one embodiment, the outcome of a digital twin simulation may also include, or only be, a deployment of data and or settings such as but not limited to at least one model, calibration data, one or more operational settings of the breathing assistance device or any operative combination thereof. For example, the at least one model may be a personalized predictive model or another model that may be used by the controller used with the breathing assistance device. Once the deployed data is received, the operation of the breathing assistance device is updated accordingly. This may involve updating firmware used by the breathing assistance device and/or the controller.

In at least one embodiment, as explained, the input to a digital twin simulation may be from a doctor/physician/healthcare practitioner, an HME (home medical equipment retailer), a health system, an EMR, a pharmacy or a payor (e.g., reimbursement) systems, via for example, the external user computing device 150b. The user may also provide inputs themselves via a software application at the user computing device 150a to indicate how they are feeling due to a medical condition, use of medication, and/or due to the sleep therapy. These inputs may then allow for simulation of the effects of the patient/user for taking certain medication so that the therapy module 124 can provide a recommendation for applying the right sleep therapy to the user. The user computing device 150a may be a smartphone.

In at least one embodiment, an output of the digital twin simulation may be halting or adjusting therapy under certain situations. For example, maxing out pressure (using high pressures) in the airflow is not appropriate for a heart surgery patient. Thus, if a user has had a surgery and are taking a medication this can be provided as input to the digital twin simulation and a decision may be made based on these inputs to alter therapy.

In at least one embodiment, different levels of data logging may be set during initialization for the digital twin simulation. For example, in the event of a patient complaint the ability to turn on more active logging may be helpful for investigating the complaint. For example, the frequency of data logging may change in the event of an investigation (e.g., if the sampling rate of data collection was 20 Hz, it may be increased to 200 Hz in this or other situations. Alternatively, or in addition thereto, other extra sensors that are available but are not normally used may be activated to provide additional data for the investigation. For example, a sound sensor that is usually not on may be activated. With the use of extra data logging the causes of complaints may be ruled out to narrow it down to the actual cause. Extra data logging may be used in other situations such as when a user is experiencing a particular medical condition, e.g., heart failure, and the extra data may be analyzed to determine whether the sleep therapy being provided by the breathing assistance device is aggravating or causing the medical condition or whether the sleep therapy can be ruled out as a cause/aggravator of the medical condition. Accordingly, performing the simulation involves obtaining an increasing an amount of data logging and analyzing the increased amount of data to investigate whether sleep therapy has caused and/or aggravated a medical condition for the user.

While the applicant's teachings described herein are in conjunction with various embodiments for illustrative purposes, it is not intended that the applicant's teachings be limited to such embodiments. On the contrary, the applicant's teachings described and illustrated herein encompass various alternatives, modifications, and equivalents, without generally departing from the embodiments described herein. For example, while the teachings described and shown herein may comprise certain elements/components and steps, modifications may be made as is known to those skilled in the art. For example, selected features from one or more of the example embodiments described herein in accordance with the teachings herein may be combined to create alternative embodiments that are not explicitly described. All values and sub-ranges within disclosed ranges are also disclosed. The subject matter described herein intends to cover and embrace all suitable changes in technology.

1. A controller for controlling the operation of a breathing assistance device that provides breathing assistance to a user, wherein the controller comprises:
a memory unit that comprises software instructions and parameters for at least one trained predictive model, the trained predictive model able to generate, based on sensor data, a nowcast of the user's current breathing state by determining a first plurality of probabilities, each of the first plurality of probabilities corresponding to a respective current breathing state of the user, and a forecast of the user's future breathing state by determining a second plurality of probabilities, each of the second plurality of probabilities corresponding to a respective predicted future breathing state of the user, within a predicted time period; and
a processor that is electronically coupled to the memory unit, the processor being configured to generate a control signal for controlling the breathing assistance device for a current monitoring time period by:
receiving the sensor data obtained by one or more sensors, the sensor data including measurements of at least one airflow parameter of the user's airflow during the current monitoring time period when the user is using the breathing assistance device;
applying the trained predictive model to generate the nowcast and the forecast;
identifying one or more false negative predictions when the sensor data corresponding to the current monitoring time period indicates a breathing event and the nowcast and/or the forecast corresponding to the current monitoring time period indicates a normal breathing state;
extracting false negative data comprising, for each of the one or more false negative predictions, the nowcast, the forecast, and a portion of the sensor data extending from a first time point before an onset of the false negative prediction to a second time point after an offset of the false negative prediction;
generating a personalized predictive model by re-training the trained predictive model using the false negative data, the personalized predictive model being personalized to the user; and
saving the personalized predictive model to the memory unit.

2. The controller of claim 1, wherein the processor is further configured to generate the personalized predictive model after a minimum number of false negative predictions are identified.

3. The controller of claim 2, wherein the processor is further configured to generate simulated false negative data for re-training the trained predictive model when an insufficient number of false negative data occurs during use by:
applying one or more signal processing techniques to the sensor data, the one or more signal processing techniques comprising: jittering, noise addition, magnitude scaling, magnitude warping, filtering, phase warping, phase scaling, or chunk truncating.

4. The controller of claim 2, wherein the minimum number of false negative predictions is in a range of one to a total number of time points at which the nowcast and the forecast are generated during a monitoring time period in which the airflow is provided by the breathing assistance device to the user.

5. The controller of claim 1, wherein the processor is further configured to:
determine one or more of a sensitivity, a precision, an F1 score and/or an adjusted F1 score of the trained predictive model, and
generate the personalized predictive model based on one or more of the sensitivity, the precision, the F1 score and/or the adjusted F1 score.

6. The controller of claim 1, wherein the processor is further configured to pre-process the false negative data using one or more of: normalization, sensor data averaging, principal component analysis, independent component analysis, down sampling, up sampling, frequency filtering, or manual inspection of the sensor data.

7. The controller of claim 1, wherein the processor is further configured to re-train the trained predictive model using the sensor data and one or more of: transfer learning, tuning one or more parameters of the trained predictive model, adding a layer to the trained predictive model, reinforcement learning, or any combination thereof.

8. The controller of claim 7, wherein the predictive model comprises one or more nodes and one or more layers, and the one or more parameters of the trained predictive model that is tunable include a type of node, a selection of nodes, a node weight, a node activation, a node memory, a number of connections between nodes, an orientation of connections between nodes, an orientation of connections between layers, a type of layer, a number of layers, a connection between layers, a number of inputs, a number of outputs, or an operable combination thereof.

9. The controller of claim 1, wherein the respective current breathing state of the user and the respective predicted future breathing state of the user comprise components including normal breathing or one or more respiratory failure events.

10. The controller of claim 9, wherein,
the one or more respiratory failure events comprise components including obstructive apnea, central apnea, central hypopnea, obstructive hypopnea, respiratory effort related arousal, an unclassified event or any operable combination thereof, and
the respiratory effort related arousal includes flow limitation, snoring, oxygen desaturation, fragmentation, heart rate abnormality, or any combination thereof.

11. The controller of claim 1, wherein,
the nowcast corresponding to the current period indicates the breathing event based on a first comparison of one or more of the first plurality of probabilities to one or more first thresholds and the forecast corresponding to the current period indicates the normal breathing state based on a second comparison of one or more of the second plurality of probabilities to one or more second thresholds, and
the one or more first thresholds and/or the one or more second thresholds are personalized to the user and adjustable.

12. The controller of claim 1 further comprising a communication module, wherein the processor is further configured to:
transmit, via the communication module, the false negative data to a remote processor that is remote located from the controller; and
receive, via the communication module, the personalized predictive model generated at the remote processor.

13. The controller of claim 1, wherein the processor is further configured to determine one or more of a pressure increase rate, a pressure decrease rate, and/or a pressure amplitude for adjusting the airflow provided by the breathing assistance device to the user based on the personalized predictive model.

14. A controller for controlling the operation of a breathing assistance device that provides breathing assistance to a user, wherein the controller comprises:
a memory unit that comprises software instructions and parameters for at least one trained predictive model, the trained predictive model able to generate, based on sensor data, a nowcast of the user's current breathing state by determining a first plurality of probabilities, each of the first plurality of probabilities corresponding to a respective current breathing state of the user, and a forecast of the user's future breathing state by determining a second plurality of probabilities, each of the second plurality of probabilities corresponding to a respective predicted future breathing state of the user, within a predicted time period; and
a processor that is electronically coupled to the memory unit, the processor being configured to generate a control signal for controlling the breathing assistance device for a current monitoring time period by:
receiving the sensor data obtained by one or more sensors, the sensor data corresponding to measurements of at least one airflow parameter of the user's airflow during the current monitoring time period when the user is using the breathing assistance device;
applying the trained predictive model to generate the nowcast and the forecast;
generating a summary representation of the user, the summary representation comprising user data;
generating the personalized predictive model by conditioning the trained predictive model using the summary representation, the personalized predictive model being personalized to the user; and
deploying the personalized predictive model on the processor of the breathing assistance device controller.

15. The controller of claim 14, wherein the user data comprises one or more of the user's weight, height, gender, sex, age, body mass index, apnea-hypopnea index, SpO2, mask type of the breathing assistance device, prescribed pressure to be provided by the breathing assistance device, location type, or location elevation.

16. The controller of claim 14, wherein the user data comprises one or more statistical representations of the user's breathing based on the sensor data, the one or more statistical representations of the user's breathing comprising an average waveform of a breath of the user, a variance for each sample timepoint within the average waveform, or one or more of a minimum, maximum, average, median, or variance of one or more of the user's air flow, air pressure, tidal volume, respiratory rate, SpO2, heart rate, sound or motion.

17. The controller of claim 14, wherein the user data comprises one or more statistical representations of the user's environment based on the sensor data, the one or more statistical representations of the user's environment comprising one or more of a minimum, maximum, average, median, or variance of one or more of temperature, ambient CO2, or ambient O2.

18. The controller of claim 14, wherein the processor is further configured to generate the summary representation by applying the trained predictive model using one or more embedding layers.

19. The controller of claim 14, wherein the processor is further configured to condition the trained predictive model using the summary representation by:
providing the summary representation as input to the trained predictive model;
adapting a feature representation based on cross-attention with the summary representation, the feature representation being based on the sensor data; or
providing the summary representation as input to a normalization block characterized by an offset factor and a scale factor for each feature corresponding to the normalization block being determined by a machine learning model conditioned on the summary representation.

20. A system for simulating one or more of an operation of a breathing assistance device and a health state of a user receiving assistance from the breathing assistance device, the system comprising:
one or more sensors for measuring sensor data;
a database storing a user model of the user, the user model being adapted for modeling one or more internal systems of the user, a device model of the breathing assistance device, the device model being adapted for modeling one or more components of the breathing assistance device, one or more subsystems of the breathing assistance device, or one or more functions of the breathing assistance device, or any operable combination thereof, and a personalized predictive model for adjusting an airflow provided by the breathing assistance device, the personalized predictive model being adapted for generating, based on the sensor data, a nowcast of the user's current breathing state by determining a first plurality of probabilities, each of the first plurality of probabilities corresponding to a respective current breathing state of the user, and a forecast of the user's future breathing state by determining a second plurality of probabilities, each of the second plurality of probabilities corresponding to a respective predicted future breathing state of the user, within a predicted time period;
a controller in communication with the one or more sensors and the database, the controller comprising at least one processor configured to:
i) receive the sensor data from one or more sensors;
ii) determine one or more of an expected state of the breathing assistance device and an expected health state of the user based on one or more of the model of the breathing assistance device the model of the user, and the personalized predictive model, and the sensor data
iii) determining a device correction factor for the breathing assistance device for improving the health state of the user, based on the expected health state of the user; and
iv) automatically adjusting the operation of the breathing assistance device according to the device correction factor.
